(19) United States
(12) Patent Application Publication
(10) Pub. No.: US 2026/0240622 A1
(43) Pub. Date: Aug. 20, 2026

(54) Methods and Systems for Augmenting a Real-Time Intraoperative X-ray Video Feed with Anatomical and Device Related Overlays and Metrics

(71) Applicants: Stryker Corporation, Portage, MI (US); Stryker European Operations Limited, Cork (IE)

(72) Inventors: Han-Wei Wang, Fremont, CA (US); Halil Karabey, San Jose, CA (US); Stephen Porter, Piedmont, CA (US); Keng-Tsai Lin, San Jose, CA (US); Nikhil Sharma, Redwood City, CA (US); Shubham Suresh Patil, Palo Alto, CA (US); Mayank Kumar, San Jose, CA (US); Anirudh Anupam Chaturvedi, Fremont, CA (US); Siddarth Satish, Portola Valley, CA (US); Nikolas Lessmann, Utrecht (NL)

(21) Appl. No.: 19/162,731

(22) PCT Filed: May 24, 2023
(86) PCT No.: PCT/US2023/067401

§ 371 Date: Sep. 5, 2025

Related U.S. Application Data
(60) Provisional application No. 63/488,655, filed on Mar. 6, 2023.

(51) Int. Cl. A61B 90/00; A61B 34/00; A61B 34/10; A61B 34/30; A61B 34/37

(52) U.S. Cl. CPC A61B 90/37; A61B 34/10; A61B 34/25; A61B 34/37; A61B 2034/105; A61B 2034/254; A61B 2034/303; A61B 2090/365; A61B 2090/376

(57) ABSTRACT

An example method includes: receiving, in real-time, a video stream of an interventional procedure captured by an image-capture device of a body lumen and a device being deployed within the body lumen; identifying, in the video stream, the body lumen and the device being deployed within the body lumen; visually presenting body lumen markers indicating characteristics of the body lumen including a curvature marking and a no-start zone that should be avoided when deploying the device; visually presenting a display of device markers indicating position of the device in real-time during deployment; and providing, on the video stream in real-time, visual indicators of parameters of the device.

### CROSS REFERENCE TO RELATED APPLICATION

The present application claims priority to U.S. Provisional patent application No. 63/488,655, filed on Mar. 6, 2023, the entire contents of which are herein incorporated by reference as if fully set forth in this description.

### BACKGROUND

X-ray angiography helps visualize body lumen (e.g., blood vessel) pathways with the body lumen regions under consideration during body lumen (e.g., endovascular) operations. As X-ray angiography continues to be one of the major components of the success of body lumen intervention, real-time clinical decision-making relies heavily on the surgeon's perception of the angiography feed and its interpretation. Such decision making is a time-sensitive, multi-dimensional process that needs real-time multi-dimensional approaches, utilizing both two-dimensional (2D) and three-dimensional (3D) ways to reliably discern multi-aspects of such intervention procedures with visual additions to the body lumens and the deployment site of devices such as low diverting stents, adjunctive stents, intrasaccular devices, coils, other embolic materials such as beads, liquids, particles etc.

It is with respect to these and other considerations that the disclosure made herein is presented.

### SUMMARY OF THE INVENTION

Within examples, described herein are methods and systems for augmenting a real-time intraoperative X-ray video feed with anatomical, 3D rendering, and device related overlays and metrics.

Within additional examples described herein, systems and methods are disclosed that relate to methods and systems to augment the real-time intraoperative X-ray video feed with several anatomical, catheter, 3D rendering, and device related overlays, while simultaneously adding predictive deployment-related guidance or metrics for operating surgeons or robotic systems.

The features, functions, and advantages that have been discussed can be achieved independently in various examples or may be combined in yet other examples. Further details of the examples can be seen with reference to the following description and drawings.

### BRIEF DESCRIPTION OF THE FIGURES

The novel features believed characteristic of the illustrative examples are set forth in the appended claims. The illustrative examples, however, as well as a preferred mode of use, further objectives and descriptions thereof, will best be understood by reference to the following detailed description of an illustrative example of the present disclosure when read in conjunction with the accompanying Figures

FIG. 1 is a block diagram of a system including an image-capture device, a device deployment module, and a display device, in accordance with an example implementation.

FIG. 1A is a block diagram showing operations performed by a device deployment module, in accordance with an example implementation.

FIG. 2 illustrates a block diagram representing the system of FIG. 1, in accordance with an example implementation.

FIG. 3 illustrates another block diagram representing the system of FIG. 1 and showing details of body lumen identification, in accordance with an example implementation.

FIG. 3A illustrates apposition mismatch of a device deployed within a body lumen, in accordance with an example implementation.

FIG. 4 illustrates a block diagram representing the system of FIG. 1 with details associated with identified parameters of a device, in accordance with an example implementation.

FIG. 4A illustrates determining coning angle among other features of a device, in accordance with an example implementation.

FIG. 5 illustrates another block diagram representing the system of FIG. 1 and showing details of body lumen identification, in accordance with an example implementation.

FIG. 5A illustrates superimposing a centerline and body lumen wall circumferential rings within a body lumen, in accordance with an example implementation.

FIG. 5B illustrates a preferred catheter track guideline, which is offset from a body lumen centerline, in accordance with an example implementation.

FIG. 5C illustrates a device being deployed within a body lumen, in accordance with an example implementation.

FIG. 5D illustrates an example of body lumen model registration between 2D and 3D, in accordance with an example implementation.

FIG. 6 illustrates a block diagram representing the system of FIG. 1 showing generation of a probability graph of a landing zone of a device, in accordance with an example implementation.

FIG. 7 illustrates linearized graphical information that can be generated by a device deployment module of the system of FIG. 1 to provide key information for body lumen intervention via a real-time video stream of an X-ray angiography, in accordance with an example implementation.

FIG. 7A a radial vector of an osculating circle at a given point and an outward deviation vector, pointing away from a center of the osculating circle, in accordance with an example implementation.

FIG. 8 illustrates an image augmented with various markers, in accordance with an example implementation.

FIG. 9 illustrates generation of a mask of a wire and deployed device using a trained model, in accordance with an example implementation.

FIG. 10 illustrates a block diagram representing a generative modeling workflow, in accordance with an example implementation.

FIG. 11 illustrates a low-dose X-ray image and a corresponding high-dose X-ray image, in accordance with an example.

FIG. 12 is a block diagram of a computing device, in accordance with an example implementation.

FIG. 13 is a flowchart of a method for augmenting a real-time Intraoperative X-ray video feed with anatomical and device related overlays and metrics, in accordance with an example implementation.

FIG. 14 is a flowchart of additional operations that are executable with the method of FIG. 13, in accordance with an example implementation.

FIG. 15 is a flowchart of additional operations that are executable with the method of FIG. 13, in accordance with an example implementation.

FIG. 16 is a flowchart of additional operations that are executable with the method of FIG. 13, in accordance with an example implementation.

FIG. 17 is a flowchart of additional operations that are executable with the method of FIG. 13, in accordance with an example implementation.

FIG. 18 is a flowchart of additional operations that are executable with the method of FIG. 13, in accordance with an example implementation.

FIG. 19 is a flowchart of additional operations that are executable with the method of FIG. 13, in accordance with an example implementation.

FIG. 20 is a flowchart of additional operations that are executable with the method of FIG. 13, in accordance with an example implementation.

FIG. 21 is a flowchart of additional operations that are executable with the method of FIG. 13, in accordance with an example implementation.

FIG. 22 is a flowchart of additional operations that are executable with the method of FIG. 13, in accordance with an example implementation.

### DETAILED DESCRIPTION

Disclosed herein are systems and methods that involve: capturing angiographic real-time video feed during body lumen (e.g., endovascular) intervention operations such as placement of devices (e.g., flow diversion devices); analyzing the real-time video feed; performing analytics on the real-time video and imaging feed to determine where a catheter, wire (e.g., guidewire), and device are located during deployment; visually presenting a display (e.g., superimposing on the video or on a separate display) in real-time body lumen markers, device (e.g., stent) boundaries and characteristics, deployment guides and predictions regarding deployment of the device; providing deployment-related quality assessment metrics; and guiding a surgeon or communication with a robotic system during the intervention.

The term “body lumen” is used here in to indicate blood vessel, lymph vessel, bile duct, esophagus, trachea or other bodily lumen. Further, the term “device” is used generally to indicate flow diverting stents, adjunctive stents, intrasaccular devices, coils, other embolic materials such as beads, liquids, particles, etc.

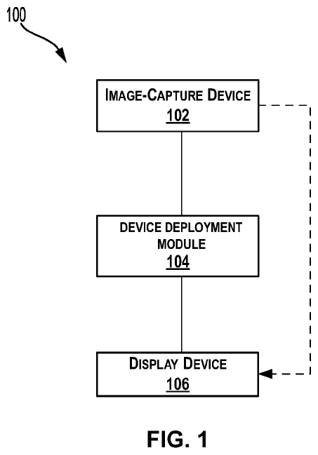

FIG. 1 is a block diagram of a system 100 including an image-capture device 102, a device deployment module 104, and a display device 106, in accordance with an example implementation. Components of the system 100 may be configured to work in an interconnected fashion with each other and/or with other components coupled to respective systems. One or more of the described operations or components of the system 100 may be divided up into additional operational or physical components, or combined into fewer operational or physical components. In some further examples, additional operational and/or physical components may be added to the system 100. Still further, any of the components or modules of the system 100 may include or be provided in the form of a processor (e.g., a microprocessor, a digital signal processor, etc.) configured to execute program code including one or more instructions for implementing logical operations described herein.

The system 100 may further include any type of computer readable medium (non-transitory computer-readable medium) or memory, for example, such as a storage device including a disk or hard drive, to store the program code that when executed by one or more processors cause the system 100 to perform the operations described herein. In an example, the system 100 may be included within other systems.

The image-capture device 102 is configured to directly capture, or read from available data streams, images and video of a body lumen during an interventional procedure (e.g., deployment of a stent such as a flow diverting device in a body lumen). For example, the image-capture device 102 would preferably include a bi-planar angiography X-ray system (e.g., the Azurion system from Philips Healthcare or the Artis system from Siemens Healthineers) or a computerized tomography (CT) scanning device that combines a series of X-ray images taken from different angles around a body of the patient and uses computer processing to generate cross-sectional images (slices) of the body lumens.

The image capture device could alternatively be a data collection device which extracts available imaging information from separate angiographic or CT equipment.

In an example, the image-capture device 102 could include a micro-CT scanning device that uses a three-dimensional (3D) imaging technique utilizing X-rays to see inside the body of the patient, slice by slice. Micro-CT scanning is similar to CT scan imaging but on a small scale with enhanced resolution. For example, body lumens can be imaged with pixel sizes as small as 100 nanometers and objects can be scanned as large as 200 millimeters in diameter.

As such, in an example, the image-capture device 102 can include an X-ray source generating X-rays that are then transmitted through the part of the patient that has a body lumen of interest. The image-capture device 102 also includes an X-ray detector that records the X-rays as a 2D projection image. The X-ray source may then be rotated a fraction of a degree on a rotational platform, and another X-ray projection image is taken. This step is repeated through a 180-degree or 360 degrees, thereby capturing images of the body lumen from different angles.

In an example, the image-capture device 102 can also generate a real-time video of the body lumen. For instance, the image-capture device 102 can include at least one camera (e.g., one camera or two cameras in a bi-planar setup) that is deployed with the body lumen device in the body lumen of the patient, and the camera generates a real-time feed of the body lumen and the deployment. In another example, the image-capture device 102 includes an external imaging device (CT scanning device) that generates a real-time video feed of a body lumen and the device being deployed inside the body lumen (e.g., stent being deployed via a catheter and wire). The image-capture device 102 can be configured to extract still images from the video. The image-capture device 102 is then configured to provide such video feed and/or images to the display device 106 to be displayed thereon. An example of such devices could be in a catheterization laboratory used in neuro, cardio, and peripheral body lumen interventions.

The device deployment module 104 is configured to receive such video, and is configured to analyze the video, and perform analytics on the video to determine where a catheter, wire, and device are in real-time during deployment of the device. The device deployment module 104 then communicates such information to the display device 106, which visually presents the information overlaid on the video or images to the healthcare professional. Particularly, the device deployment module 104 may visually superimpose on the images and videos on the display device 106 device (e.g., stent) and body lumen markers, guides and guidelines, and predictions regarding deployment of the device, and may provide deployment-related quality assessment metrics (e.g., scores) on the display device 106. Such visual information and metrics may offer guidance to a surgeon or direct a robotic system to adjust deployment techniques to enhance the deployment of the device and achieve a desired outcome.

As such, the device deployment module 104 is configured to receive a digital video feed from an imaging system, such as X-ray angiography and CT scanning device, during an interventional procedure to intraoperatively, and in real-time, display information to the physician or the operator that helps the physician or operator position a body lumen device in an optimal configuration. The term “intraoperative” is used here to indicate occurrence or performance during the course of a surgical operation. In an example, the device deployment module 104 augments the information on the video feed, and displays the augmented video feed or images extracted therefrom on the display device 106.

A flow diverting stent is used throughout herein as an example device.

However, it should be understood that the techniques, methods, and systems disclosed herein can be used with other interventional devices (e.g., embolization coiling, intrasaccular device deployment, peripheral body lumen or other luminal such as carotid, biliary or femoral stent deployments, etc.).

In an example, the stent can be mounted or sheathed within a catheter, which is pushed over a wire to a location in the body lumen where the stent is to be deployed or positioned. Example augmented information generated by the device deployment module 104 include: (i) identification and marking of where the stent, catheter, and wire are, and (ii) anatomical identifications and catheter landmarks to add visual context and enhance scene understanding for the physician or the operator, particularly when using a low-dose X-ray system that produces lower contrast X-ray images. By providing increased clarity of the stent, catheter, and guide wire, and superimposing relevant features (anatomical, physiological, or otherwise), the device deployment module 104 may diminish the need for high-dose X-ray imaging and reduce the need to for several intra-arterial injection of the contrast agent to generate digital subtracted angiography “roadmap” images., thereby reducing exposure to contrast agent and reducing the required computational power, while increasing surgical efficiency and precision.

In an example, the information could also include metrics of surgical scoring. For instance, the device deployment module 104 could provide a measure indicative of how well the surgical procedure/deployment happens with respect to the pre-operative surgical plans or expected deployment outcome, or predictive deployment status to help support correct deployment.

In examples, the video augmentation can be done directly on the display device 106, which can be placed in a neuro intervention catheterization lab. In another example, the display device 106 can include an augmented reality or virtual reality headset that can overlay the information on top of existing displays or on a virtual display, thereby reducing the need for extra hardware in a catheterization lab.

In another example, video and data augmentation can be presented using a hologram as the display device 106, which projects 2D information and 3D models in the space close to the user without the need of wearing additional visual devices on the user. The holographic visual information can be operated interactively to switch and alter the information and models by the user's commands perioperatively.

As such, the device deployment module 104 is configured to perform operations including feature recognition of vasculature and devices being deployed, analyzing and scoring in real-time, and provide various outputs and predictions to a user or a robotic system.

FIG. 1A is a block diagram showing operations performed by the device deployment module 104, in accordance with an example implementation. As depicted, analysis of deployment of a device, e.g., a stent, starts with feature recognitions, such as wires, markers, body lumens, devices, curves, diameters, lengths, sizes, references, centerline, wire/strut braid angle, etc.

The recognized or identified features can be used to derive multiple inputs including coning angle, distance between markers, curvature of the body lumen, curvature of the stent, apposition of the stent with the body lumen, location of the stent, shape of the stent, irregular shapes, which are not clinically desired (e.g., ribboning, twisting, fish-mouthing), deviation from the centerline, deviation from ideal or define deployment path, braid angles, or braid density. Distance between markers can also include the distance from a particular marker to a point of reference on a body lumen, landmarks such as aneurysm opening, beginning and end of a curve, ideal landing zone, ideal or defined path, deviation from the centerline, etc.

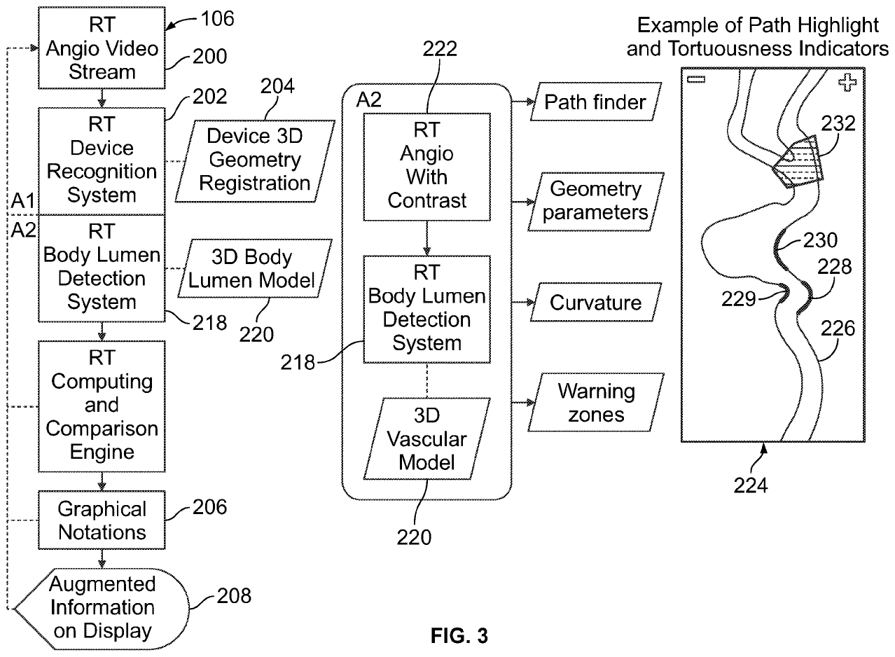

Coning angle is not a fixed angle but varies during the deployment procedure. The ideal coning angle is a function of body lumen tortuosity, curvature, amount of tensile or compressive force applied to the catheter, the diameter of the native body lumen, aneurysm dimensions, the net force between the microcatheter and the stent delivery wire. An example of how coning angle is determined is described below with respect to FIG. 4A.

Curvature of the body lumen can also be determined and is an input to the ideal deployment calculation. A more curved body lumen presents additional challenges for deployment and is likely to require more push-pull actions and catheter manipulations to achieve the desired result. The curvature is also an important, lateral input for analyzing cone shape, corning angle, centerline deviation, deployment path, and so on.

Braid angle and braid density can be partially recognized as a stent feature, or can be applied to the stenting area via known and recognized info such as stent design, curvature, diameter, apposition, etc. The braid angle and braid density are factors for the prediction of flow diversion.

Apposition of the stent with the body lumen is also valuable in assessing deployment techniques, positioning, landing zone, foreshortening, avoiding endoleaks, stent packing, etc.

In addition, feature recognition can identify actions and events during the intervention case. Actions and events could be detected and used to improve on the scoring algorithm of stent deployment. For example a contrast run/flush could be used to determine any areas of stent mal-apposition, resheating of the stent could be captured to improve technique or detect any slippage of stent from the resheath pad, movement of the delivery wire of the stent into smaller branches of arteries could alert the interventionist, etc.

With the inputs from the feature recognition to the analysis and scoring, outputs and predictions generated from the system 100 (e.g., as shown in FIG. 1A) can be presented, notified, displayed, or messaged as results to the user.

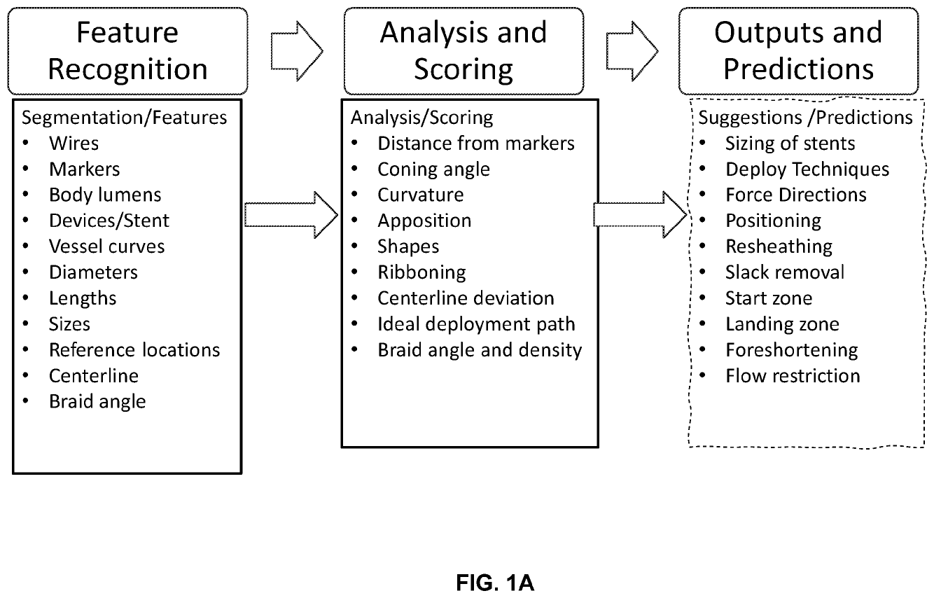

FIG. 2 illustrates a block diagram representing the system 100, in accordance with an example implementation. Block 200 represents real-time acquisition of a data stream including video and other information related to a body lumen (angio) in which a body lumen device such as a stent is being positioned. The data stream is shown on the display device 106 (see augmented image 210). The data stream, including video and other information, can be an X-ray angiography data stream captured by a CT scanning device (the image-capture device 102 described above), for example. The video stream could also be fetched from digital video feeds of the imaging system (such as High-Definition Multimedia Interface (HDMI) or Digital Visual Interface (DVI), Serial ports, etc.), or from external screen captures of the X-ray angiography (e.g., an external camera pointing at a screen in the catheterization lab showing the video feed).

The video feed is considered as an input stream to block 202. The block 202 can be implemented by the device deployment module 104, for example. At the block 202, the device deployment module 104 can identify via image recognition techniques the body lumen device (e.g., a stent) that is traversing the body lumen via catheter and wire.

As an example, the device deployment module 104 can have access at block 204 to a device 3D geometric registration (e.g., a 3D model of the stent) in 3D space. As such, the device deployment module 104 can identify location of the stent, stent markers, position and orientation of the stent, the catheter, and the wire. Particularly, the device deployment module 104 can identify features of the stent (e.g., proximal end, distal end, wire/strut angels, orientation, etc.).

Such information (locations, markers, features, indicators of the stent) are generated as graphical notations at block 206. The graphical notations are then augmented at block 208 onto the video feed on the display device 106. Both the video stream acquired at the block 200 and the graphical annotations of the block 206 can be displayed on the display device 106. In other words, the graphical annotations are superimposed or augmented onto the video stream while being displayed on the display device 106.

The augmented image 210 illustrates information associated with a stent, catheter, and wire augmented onto the video feed. The image shows a stent 212 and a wire 214 that could be distinguished or clarified in the augmented image 210 via color coding or contrast enhancement.

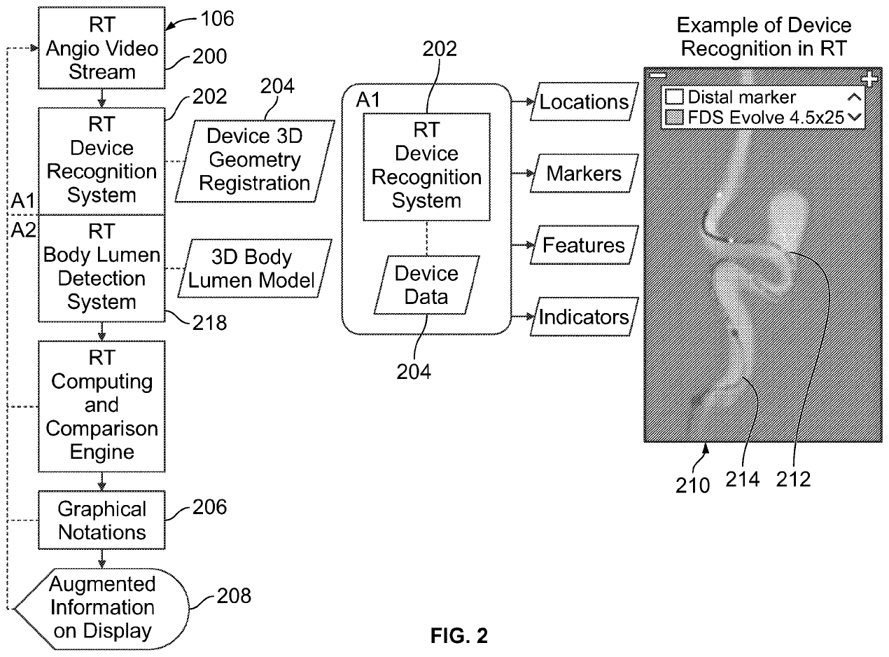

In addition to the block 202 associated with real-time recognition of the device (stent) being deployed, the device deployment module 104 can detect at block 218 features of the body lumen in which the device is being deployed. Based on such detection, the device deployment module 104 can recognize features of the body lumen, and it can identify different regions of the body lumen and their suitability for deployment of the stent. The device deployment module 104 can also generate and/or point out indicators associated with the body lumen such as a tortuosity indicator, a no start zone indicator, etc. as described in more details next with respect to FIG. 3.

FIG. 3 illustrates another block diagram representing the system 100 and showing details of body lumen identification, in accordance with an example implementation. The block 218 can be implemented by the device deployment module 104, which can have access to a 3D body lumen model at block 220. For example, the device deployment module 104 can have access to a 3D model of the body lumen generated from images collected prior to the intervention procedure, for example, via the image-capture device 102 (e.g., a rotational scanning angiography system or CT scanning device).

Further, a contrast agent can be flushed through the body lumens while capturing the video feed. The device deployment module 104 can have access via the video feed to images at block 222 when the contrast agent is flushed, and the device deployment module 104 can then map the body lumen pathways (e.g., details of the body lumen including branches, etc.). The device deployment module 104 can also compare the 3D body lumen model of the block 220 to the body lumen pathways mapped when the contrast agent is flushed through the body lumens at the block 222 to identify with visual clarity different sections of interest in the body lumen. Such comparison may also be useful for spatial registration (e.g., alignment) of the 3D body lumen model of the block 220 to live images.

For example, as depicted in FIG. 3, the device deployment module 104 can identify a path for the catheter, wire, and stent within the body lumen (path finder), can identify geometric parameters of the body lumen such as diameter of the body lumen at various sections, can determine curvature/tortuosity of the body lumen at different sections, and can also identify zones of the body lumen where a stent should not be deployed (warning zones). The device deployment module 104 can then visually superimpose body lumen markers (e.g., identifying markers showing luminal extents, centerlines, boundaries, curvature, etc.) on the video feed images.

FIG. 3 illustrates an augmented image 224 showing an image from the video feed depicting a body lumen 226. The device deployment module 104 can determine tortuosity or the curvature of a body lumen 226 accurately. The device deployment module 104 can further identify and label in the augmented image 224 the most tortuous body lumen regions and their properties, including curvature in 3D, body lumen loop, or branches in 3D space. For instance, the device deployment module 104 can superimpose in the augmented image 224 a tortuousness indicator. Particularly, the device deployment module 104 can mark the body lumen 226 with curvature markings such as curvature marking 228, curvature marking 229, and curvature marking 230 indicative of regions of the body lumen 226 with curvature exceeding a particular threshold curvature. In this context, curvature can, for example, be determined as an inverse of a radius of a portion of the body lumen 226 and operates as an indicator of how tortuous the body lumen 226 is. The curvature markings 228-230 can, for example, be marked and indicated in a distinctive color (e.g., red) to visually clarify such markings to the surgeon. These markings can help a surgeon determine where to deploy the stent and which regions to avoid during deployment.

Further, the device deployment module 104 can also identify no-start and no-end zones in the body lumen 226. No-start zones are regions with unfavorable tortuosity, hidden branches, or bifurcations that might compromise performance of the stent if the distal end of the stent is disposed in such regions. In other words, the surgeon should avoid having the distal end of the stent disposed in such no-start zones when deployment is beginning and should avoid having the proximal end of the stent disposed in at no-end zones when deployment is finished. The no-start and no-end zones could also be determined and manually input by the surgeon prior to deploying the stent.

As shown in FIG. 3, the device deployment module 104 marks a no-start zone 232 where the body lumen 226 bifurcates. On the augmented image 224, the device deployment module 104 places a polygonal shape around the no-start zone 232 to point out such zone to the surgeon during deployment of the stent. Alternatively a no-start/end zone or a optimal start/end zone could be indicated by the body lumen circumference indicators (ovals) which are color coded to indicate good and bad areas for stent location. Alternatively, optimal start and end (“start-here” and “end-here”) zones could also be employed wherein markings are directed towards the locations that the stent would optimally start and/or end. Those locations may be determined by the device deployment module 104, input manually by the surgeon or determined by a separate planning software and input into the device deployment module 104.

Additionally, in examples, the device deployment module 104 can provide warnings, numerical scores, and other such indicators on the augmented image 224 to assist with the deployment of the stent in real-time. The awareness of tortuosity of the body lumen 226 and the no start zones may help the surgeon achieve an enhanced deployment outcome.

For example, it is desirable to have the stent be well apposed against the wall of the body lumen 226. Apposition of the stent refers to how closely the exterior peripheral surface of the stent interfaces with the inner wall of the body lumen 226. If the outer diameter of the stent is smaller than the inner diameter of the body lumen, the stent can be characterized as having loose appositioning on (mal-opposed to) the wall of the body lumen 226. Such loose appositioning might not be desirable as it could lead to migration or movement of the stent once deployed within the body lumen 226. It is rather desirable to have the stent with high apposition such that the stent is as close as possible to the wall of the body lumen to be stable in its position within the body lumen and to provide effective blood flow diversion.

Apposition can be represented by a coverage percentage at a particular cross section of the body lumen 226. For example, at a given cross-section of the body lumen 226 based on a 3D registered body lumen model, the Apposition Mismatch percentage can be determined as follows:

$$Apposition Mismatch \% = ( \frac{Cross Sectional Area of the Vessel - Cross Sectional Area of the Stent}{Cross Sectional Area of the Vessel} ) \times 100$$

In another example, the Apposition Mismatch % can also be determined as:

$$Apposition Mismatch \% = ( \frac{Local Diameter of the Vessel - Local Diameter of the Stent}{Local Diameter of the Vessel} ) \times 100$$

Other apposition metrics could be used. In an example, the wall apposition mismatch may be calculated based on the edge detection of the stent and compared with the body lumen wall from the digital subtraction angiogram image.

FIG. 3A illustrates (in a linearized manner) apposition mismatch of a stent deployed within a body lumen, in accordance with an example implementation. FIG. 3A shows a potential scenario that may occur in different underlying disease states such as Moya Moya and others. The wall apposition score depends on the percentage of stent in visual/perceived contact with body lumen wall. The risks of a higher mismatch are a potential endoleak, aneurysm recanalization or other complications related to bad wall apposition of the stent

As another example, a total apposition score could be determined for the stent. In FIG. 3A, L_{M} is a local mismatch length, LT is the overall length of the stent. The total apposition score A_{Total} can be determined as:

$${A}_{Total} = 1 - \frac{{\sum }_{1}^{n} {L}_{M , n}}{{L}_{T}}$$

The total apposition score and the apposition mismatch can also be highlighted on an augmented image to provide apposition warnings, deployment technique suggestions, or indications of potential stenting issues such as recanalization, endoleak of stent deployment, or other concerns.

Referring back to FIG. 3, at tortuous areas such as zones marked by the curvature markings 228-230, the stent might have poor apposition. Such areas ideally should be avoided, and thus such markings assist the surgeons during deployment. In some instances high curvature areas must be traversed during stenting and the markings can provide a warning to the surgeon to be more attentive to the stent apposition in those areas.

FIG. 4 illustrates a block diagram representing the system 100 with details associated with identified parameters of a stent, in accordance with an example implementation. As shown in FIG. 4, the device deployment module 104 can have access at the block 204 to 3D rendering data of a stent (e.g., a 3D model of the stent supplied by a manufacturer of the stent). The device deployment module 104 can thus superimpose the model of the stent within the body lumen and determine characteristics of the stent (e.g., apposition).

For example, as shown in FIG. 4, the device deployment module 104 can determine information including localization (positioning of the stent) within the body lumen, apposition, a centerline of the body lumen and the stent, wire orientation of the stent if the stent is a braided stent for example, coning angle, and braiding angle (e.g., half of the angle made by crossing filaments in the braid of a braided stent). The device deployment module 104 can then superimpose indicators of such information on images of the video feed.

For example, as depicted in FIG. 4, augmented image 234 depicts recognized features such as outline of a body lumen 236 superimposed on the video feed. The device deployment module 104 can further superimpose a predicted rendering 237 of a stent 238 showing a centerline 240 of the stent 238.

Further, in an example, as shown in FIG. 4, the device deployment module 104 visually superimposes on an augmented image 242 apposition indicators indicating how well the stent 238 is opposed to the wall of the body lumen 236. For example, as shown in FIG. 4, the device deployment module 104 can generate a display of ovals such as oval 244 at different sections along a length of the stent 238. The ovals are meant to indicate a relationship between a diameter of the stent 238 and a respective diameter/circumference of the body lumen 236, but appear as oval due to angularity of the body lumen 236 and the stent 238.

The oval 244 operates as an apposition indicator (e.g., how well the stent 238 is opposed against the wall of the body lumen 236) at a particular location or cross-section of the stent 238 and the body lumen 236. The ovals can be color coded. For example, green ovals can indicate acceptable opposition while red portions can indicate mal-apposition or unacceptable apposition. For example, a portion of the oval 244 can be in green, while a portion 246 can be in red to indicate mal apposition or mismatch between the body lumen 236 and the stent 238 (e.g., a diameter of the stent 238 may be greater than a diameter of the body lumen 236 at a given section).

FIG. 4A illustrates determining coning angle among other features of a stent, in accordance with an example implementation. Particularly, FIG. 4A provides an example of identifying coning angle, deployment cone shape, inner curvature side, and calculated distance from the last recognized apposition location to the catheter marker.

As an output, for example, the coning angle could be color coded in an image 247 to show the analyzed result of the deployment status. Scoring and suggested deployment techniques could also be displayed to guide the real-time deployment process. In one example, the coning angle can be recognized based on the angle formed between the marker and a defined distance. In another example, the coning angle could be calculated based on the segmentation of the identified stent at a defined distance, D, from the microcatheter marker. If “r” is the radius of the opening stent at the distance D, together, the coning angle can be calculated as: 2×arctan(r/D) on a straight body lumen, for example.

When a stent is opening at a curve, the coning angle can be defined by a hydraulic mean angle, a normalized function, or a symmetric equivalent angle from the identified segmentation of the stent. As another example, the cone shape of the tapering part of the stent can be identified and referred to the known stent response profile from force interactions. The output from the analysis could be displayed as a deployment force indicator for deployment technique suggestions. The cone angle, inner curvature, and the centerline distance from the last apposition location can also be analyzed to indicate poor or good techniques in the deployment procedure, for both opening and resheathing. Technique suggestions can also be provided by indicators or messages, including visual, audio, or audio-visual as the output to the user. Suggestions and scoring can be displayed to guide and help the procedure.

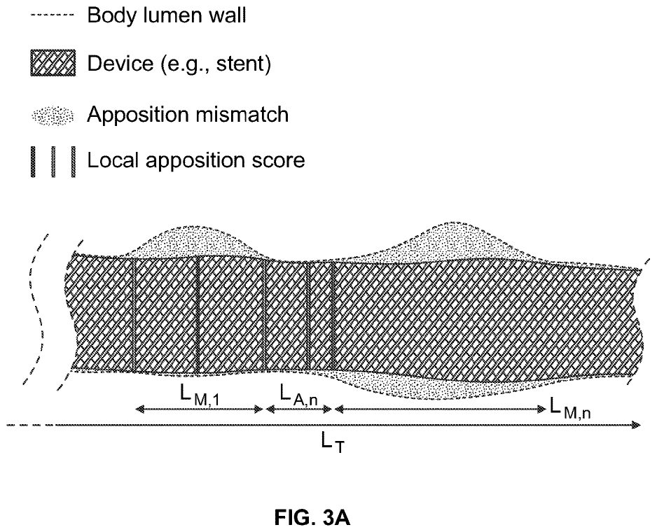

FIG. 5 illustrates another block diagram representing the system 100 and showing details of body lumen identification, in accordance with an example implementation. FIG. 5 is similar to FIG. 3 where the device deployment module 104 at the block 218 determines several parameters, such as a diameter, centerline, curvature, and narrowing indicators, of a body lumen. The device deployment module 104 then visually superimposes information or indicators of the body lumen parameters on an image of the video feed such as augmented image 248.

As shown in the augmented image 248, the device deployment module 104 identifies body lumen 250, and marks various parameters thereof. For example, the device deployment module 104 indicates a centerline 252 of the body lumen 250 as a dashed line that follows the body lumen 250 as it curves and changes its diameter. The device deployment module 104 also superimposes tortuousness indicators or curvature markings such as curvature marking 254 and curvature marking 256 indicative of regions of the body lumen 250 with curvature exceeding a particular threshold curvature.

The device deployment module 104 also identifies and marks a no-start zone 258 in the body lumen 250. As mentioned above, no-start zones are regions with unfavorable tortuosity, hidden branches, or bifurcation that might compromise performance of the stent if the distal end of the stent is disposed in such regions. Additionally or alternatively, the device deployment module 104 can identify “start-here” and “end-here” zones that could be optimal for starting and ending a stent within the body lumen 250.

As such, the device deployment module 104 performs real-time angiography registration of various features of the body lumen 250 to assist in optimal outcome of stent deployment. No-start zone, centerline, tortuosity, and other information are superimposed on the real-time angiography screen (the display device 106) to provide visual identification of various features of the body lumen and the stent.

FIGS. 2-5 illustrate examples of the real-time deployment analysis performed by the device deployment module 104 that can provide animated or augmented graphical features and indicators to highlight the prominent features of a body lumen and a stent on the X-ray angiography. When the device deployment module 104 identifies and registers (determines position and orientation of) a stent, the device deployment module 104 then labels the various stent features. The displayed output (augmented image) on the display device 106 could include real-time location, centerlines, circumferential rings or loops (which may appears as circles, ovals, intersections of two or more circles, or ovals or distorted forms thereof) denoting or defining wall of the body lumen, stent features, stent apposition, and critical angles of the stent.

FIG. 5A illustrates superimposing a centerline and body lumen wall circumferential rings within a body lumen, in accordance with an example implementation. As shown, the centerline of the body lumen is marked with a dotted or dashed line. The depicted body lumen wall circumferential loops or rings are defined by the intersection of the body lumen wall and a normal plane to the centerline at given point along the centerline, wherein points of origin for the rings are points of interest and are preferably spaced at equal lengths along the centerline within the region of interest for stent deployment and/or determined by points of particular interest such as the optimal stent start-here and end-here locations, the distal-most and most-proximal extents of the aneurysm ostium, the centroid of the aneurysm ostium, etc. The rings can be used as markers as well. For instance, the rings may use color coding, density of spacing, thickness of lines, opacity, or transparency to indicate various markings such as curvature, no-start/end zones, anticipated ending location, landing probability, stent wall apposition, a performance metric or score, etc.

In an example, the displayed indicators can be toggled as desired by the surgeon for deployment assistance. The determined stent features are superimposed on the specific anatomical, physiological, and physical circumstances of the body lumen to assist the surgeon in in real-time.

Further, in examples, the device deployment module 104 can determine scores or heat map-based identifiers that provide a quality assessment of the deployment of the stent and might provide warnings. An example score may include a weighted average of several parameters of deployment in real-time. For instance, the score may include a weighted average of parameters indicative of coning angle, centerline deviation, proximity of an end of the stent to a no-start or no-end zone, apposition of the stent, braid angle, deployment conditions such as undeployed, partially-deployed, under-deployed, or optimally-deployed, etc.

For example, a deployment score can be calculated as follows:

$$Deployment Score = \frac{( {w}_{1} \times C + {w}_{2} \times A + {w}_{3} \times F + {w}_{4} \times L + {w}_{5} \times D + {w}_{6} \times M )}{6}$$

where w₁-w₆ are weights assigned to specific parameters or variables during deployment, C is coning angle, A is a wall apposition score, F is a score of fish mouthing of distal end of stent, L is a landing zone score based on stent end proximity to or overlap with a no-start/no-end zone or a start-here/end-here zone, D is deployment condition based on percentage of stent deployed, and M is a deviation from a centerline of the body lumen (e.g., measured as a score. These factors are an example for illustration only. More or fewer factors can be used to determine the deployment score.

Thus, device deployment module 104 is configured to provide real-time quality assessment of the deployment. The output of the device deployment module 104 can include an overall quality score alongside a visual superimposition of the expected final position of the stent. The score changes in real-time as the surgeon adjusts positioning and deployment of the stent during the procedure.

As another example of feedback that can be provided to a user, the device deployment module 104 can provide tracking of a catheter tip during deployment. Particularly, the device deployment module 104 can track instantaneous deviation of the catheter tip from a pre-defined preferred catheter tip track guideline.

FIG. 5B illustrates a preferred catheter track guideline 257, which is offset from a body lumen centerline 259, in accordance with an example implementation. The preferred catheter track guideline 257 defines a path along which the stent deployment catheter would preferably follow if the stent is being deployed in a manner consistent with an optimal deployment plan generated by the device deployment module 104.

If the catheter is generally following the preferred catheter track guideline 257, visual and/or audio feedback can be provided to the user to indicate on-track performance. Conversely, if the catheter is generally deviating from the preferred catheter track guideline 257, a visual and/or audio feedback can be provided to the user to indicate the deviation and suggest or cue potential corrections to the user or robot to alter the deployment technique and/or update the deployment planning.

FIG. 5C illustrates a stent 249 being deployed within a body lumen 251, in accordance with an example implementation. The illustration of FIG. 5C depicts acceptable stent-wall apposition as indicated by an outline of the stent 249 meeting the extent of the body lumen rings of the body lumen 251. FIG. 5C also shows acceptable wire-catheter deployment ratio as indicated by a catheter tip being in alignment with a preferred catheter track guideline 255.

FIG. 5D illustrates an example of body lumen model registration between 2D and 3D, in accordance with an example implementation. FIG. 5D shows an X-ray image 261 on the left and a generated 3D body lumen model 263 on the right. The information can be processed at the block 218 in FIG. 3 (e.g., the real time body lumen detection system), for example. The 3D body lumen model 263 could be leveraged in real-time to accurately measure body lumen diameters and tortuosity, position and orientation of the stent and wire. This information can then be fed to the device deployment module 104.

With a bi-planar image-capture system, positions of interest identified in the video feed (such as a catheter tip, etc.) could be projected back into 3D space by knowing the X-ray source location and pointing vector and then tracing back the path that the X-ray took from X-ray source to the detector. The intersection of two such rays from a bi-planar video feed would yield coordinates in 3D space that could be related to the 3D body lumen model 263 by means of image registration (aligning images from different sources or time-points, for instance by minimizing a cost function that expresses the similarity of the images).

Further, geometrical changes of the body lumens, due to the interventional interactions, can be observed from 2D angiographic views. The changes can be superimposed into the 3D body lumen model 263 for real-time reference. Additionally, utilizing new data sets from multiple 2D views (e.g., the X-ray image 261) taken from angularly-separated imaging vectors, the changes can be modeled based on reconstruction from the new data set and based on the learning from a previously reconstructed 3D model. Updates and modifications of the body lumen model can be generated and fed into the latest 3D model to increase the accuracy of output predictions or to boost precision of measurements. The updated 3D model can then be used to update data used for marking, feedback and displays such as curvature, centerlines, catheter track lines, rings, no start/no end zones, landing zone, landing probability, apposition, etc. The 3D body lumen model 263 can be presented in an additional window or panel to enable the 3D perception and real-time 3D identification during the intervention. Such changes, for example, can be visualized directly in 3D using devices such as visual displays, hologram devices, virtual/augmented reality wearables or headsets, visual dashboard on screen, etc.

On the display channel of the 3D body lumen model 263, independent motions of user's choice or dependent rotations associated with bi-plane views can be applied. For example, motions, such as combinations of roll, yaw, pitch, and other view changes to the 3D body lumen model 263 (the displayed model) can be implemented based on the user's preference and/or based on the motion of the bi-plane gantry of the imaging system. The interactive motions on the 3D model can further assist the understanding of the case in the intervention and deployment procedure.

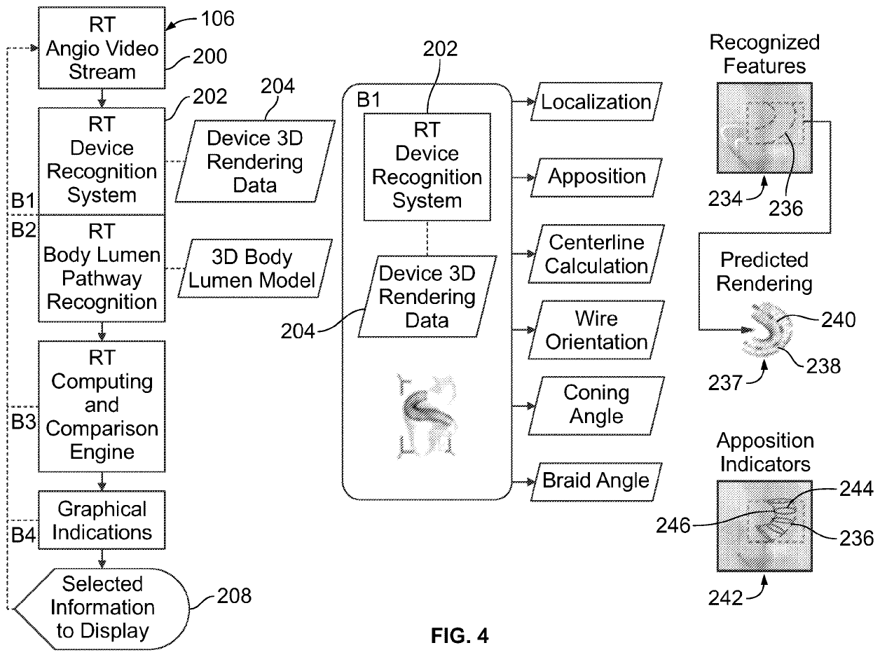

FIG. 6 illustrates a block diagram representing the system 100 showing generation of a probability graph of a landing zone of a stent, in accordance with an example implementation. The device deployment module 104 can include a real-time computing and comparison engine at block 260 configured to determine and provide graphical indicators and animation for stent deployment parameters, which assist with the understanding of the stenting effect or the deployment outcome.

In the example shown, an augmented image 262 shows a real-time landing probability chart 264 (e.g., a distribution probability curve) displayed to help predict a landing zone 266 (where a proximal end of the stent would be disposed when deployment is completed), which can be important to the outcome of the deployment.

In determining the landing zone 266, the device deployment module 104 can take into consideration effects such as foreshortening effects. A technical problem that occurs when using a stent in intrabody lumen procedures is the difficulty of predicting the final positioning of the stent after deployment inside the body lumen due to the change in length of the stent, which is dependent on the anatomy of the patient and the positioning of the stent within a body lumen. In other words, an issue of deployment of stents is the change in total length (foreshortening) that the stent experiences when the stent is released in the body lumen from a catheter. The device deployment module 104 takes foreshortening effects into consideration when determining the final length and landing zone of the stent once deployed.

While on a 2D image a particular length of a stent may appear to be sufficient, that length might not be sufficient when the stent is actually deployed in the body lumen due to foreshortening. Particularly, due to the 3D configuration of the body lumen, the body lumen might be longer than how it appears in a 2D image. For example, the body lumen may be tortuous and may have twists and bends in 3D space such that the actual length of the body lumen is longer than what appears from the perspective of a 2D image. Further, the stent may be compressed, elongated, or otherwise changes its length upon deployment due to deployment technique and/or interaction with the body lumen. The device deployment module 104 estimates such foreshortening effect, and determines a probability of the final length and landing zone of the stent as illustrated by the real-time landing probability chart 264.

In examples, based on determinations of the landing zone 266 and other characteristics of the stent, the device deployment module 104 may suggest catheter motions that results in a favorable outcome. Suggested catheter motions include adherence to a preferred catheter tip track guideline, rotation, translation, and other similar maneuvers of the catheter, microcatheter, and wire. They can also include repeated small motions or oscillations, such as linear back and forth motions, and rotational clockwise and counterclockwise repetitions. Such suggestions may be provided to a human surgeon to adjust deployment technique or can be provided as an algorithm to a robot deploying the stent.

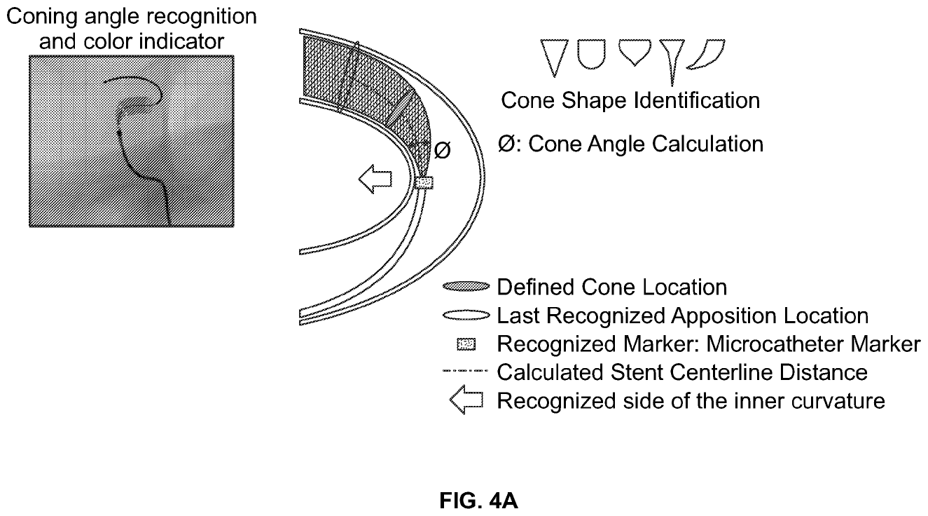

FIG. 7 illustrates linearized graphical information that can be generated by the device deployment module 104 to provide key information for body lumen intervention via a real-time video stream of an X-ray angiography, in accordance with an example implementation. By extracting geometrical information from a video feed, the device deployment module 104 can label and register the body lumen pathways and can determine information such as body lumen curvature, diameter, and centerline as described above. 3D body lumen models from the patient medical data can be co-registered to the 2D image frames extracted from the video stream to increase the accuracy of the geometrical parameters. When receiving the stent deployment video stream, the device deployment module 104 determines geometrical comparison to derive information such as deployment ratio, apposition information, catheter track-line, etc. to enable providing intraoperative procedural suggestions, scorings, or warnings.

A graph 300 shown at the top of FIG. 7 illustrates a curve 302 showing variation of curvature of a body lumen along a length of the body lumen. Graph 304 illustrates planned or predicted stent deployment location and diameter. Particularly, in the graph 304, bars such as bar 306 represent diameters of the body lumen along a length of the body lumen, corresponding to the curve 302 of the graph 300.

Also, in the graph 304, the device deployment module 104 provides a predicted or suggested location and diameter of a stent at region 308 depicted with crisscrossing lines. The region 308 is the space to be occupied by the stent. As shown, the planned location of the stent avoids starting and ending at areas of large curvatures in the body lumen as indicated the curve 302.

Graph 310 further provides stent deployment data that might be helpful to the surgeon (or robot) to achieve the suggested location of the stent. For example, the graph 310 may include graphical indicators of a deployment ratio at particular locations along the body lumen as represented by dashed line 312.

Deployment ratio refers to a wire push-to-catheter pull back ratio to obtain an optimally deployed state. Particularly, during deployment, as mentioned above, a surgeon or robot may perform oscillating movements between the wire and the catheter to un-sheath the stent from the catheter and re-sheath the stent in back and forth movements to deploy the stent in a desired configuration (at a desired location, with desired diameter, desired angle, desired body lumen wall apposition, etc.). The device deployment module 104 can provide such information to the surgeon or robot to achieve the desired deployment of the stent.

An additional indicator for improving and or monitoring deployment accuracy can take the form of a preferred catheter tip track guideline as mentioned above. A preferred catheter tip track guideline is a 3D curve that traces a preferred intra-luminal path traversing the body lumen segment along which the stent is to be deployed, which further indicates the radial deviation off the body lumen centerline along which the catheter should preferably follow during the process of stent deployment. For instance, the track guideline may correspond to the path the catheter would follow if catheter-wire manipulation is in exact adherence to the aforementioned deployment ratio. Conversely, the catheter-wire deployment ratio may be derived from the anticipated device manipulations needed to follow the track guideline.

In general the deviation of the track guideline from the centerline at a given point along the centerline would fall on or near the radial vector of an osculating circle associated with that point. FIG. 7A illustrates a radial vector 322 of an osculating circle 324 at a given point 326 and an outward deviation vector 328, pointing away from a center of the osculating circle 324, in accordance with an example implementation.

The extent and direction of the radial deviation would be determined by an analytical/geometric algorithm or a machine learning derived model, which incorporates geometric and clinical factors such as, but not limited to, the type of stent, body lumen diameter, centerline curvature at the point, the location and size of the aneurysm ostium, clinical preference for increased or decreased stent mesh density in specific locations, presence of a side branch body lumen and/or the axial proximity to the beginning and end of the deployed stent.

Further, the device deployment module 104 may update its optimal stent deployment plan, and thus alter the track guideline during the course of stent deployment to accommodate changing location of the calculated landing zone and its proximity to or overlap with the no-end zone. If the device deployment module 104 cannot find a deployment plan which avoids the no-end zone, it can warn the surgeon that the landing zone and the no-end zone are coinciding, and that re-sheathing and re-positioning of the stent is recommended.

Prior to final deployment or after complete deployment, the device deployment module 104 can generate a graph 314 for post deployment analysis. The graph 314 is similar to the graph 304 except that rather than a predicted placement of the stent, the graph 314 shows actual placement of the stent. As shown, the graph 314 can point out, emphasize or highlight regions such as region 316 where apposition is not optimal (e.g., diameter of the body lumen is larger than respective diameter of the stent at such region or vice versa). The region 316 may correspond to the portion 246 of the oval 244 as shown in FIG. 4 described above, for example.

The graph 314 also points out that the in the final deployment of the stent, the stent is shorter than predicted as indicated by unoccupied regions such as region 318 at the proximal end of the stent and region 320 at the distal end of the stent. The surgeon can then determine whether such deployment is acceptable or in the instance where final deployment has not yet occurred, whether redeployment is required.

In examples, the device deployment module 104 may involve using a neural network model that can be trained to accurately localize the various markers seen during the deployment of the stent such as the distal, proximal, and re-sheathing markers on the delivery wire, the micro-catheter, and intermediate-catheter markers.

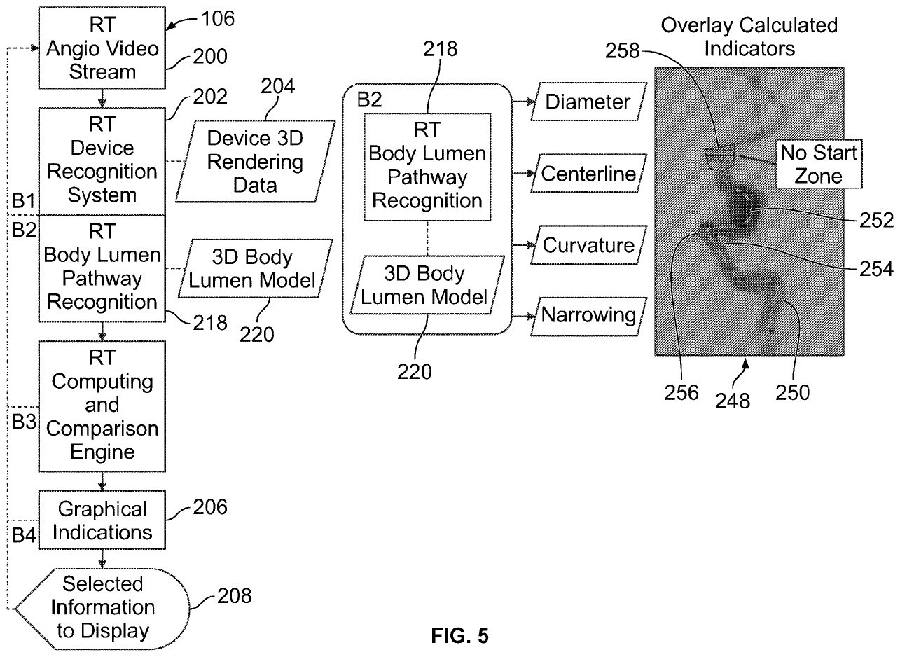

FIG. 8 illustrates an image augmented with various markers, in accordance with an example implementation. In FIG. 8, image 400 on the left illustrates a raw image from the video feed, which then input to the device deployment module 104. Thus, the image represents an input frame to the device deployment module 104. The image 400 shows a body lumen 402 and a wire 404.

The device deployment module 104 is configured to generate an augmented image 406 with marker overlaid on the image 400 (the input frame). For example, as indicated by legend 408, the device deployment module 104 marks a re-sheathing location 410, a distal marker 412 of the delivery wire, location of a microcatheter marker 414, an intermediate catheter marker 416, and a proximal marker 418 of the delivery wire.

The knowledge of the location of these markers can aid the localization and pose recognition/registration of the overall catheter device, while also assessing any inconsistencies for a given deployment. The location of the markers can be displayed in the augmented image 406 for surgeons to comprehend the location of key components of the catheter.

A machine learning model (such as a neural network model) of the device deployment module 104 can be trained directly using pre-recorded videos of body lumen procedures in a supervised or semi-supervised manner, where markers and other key points can be labeled by human experts to train a model to predict them. The neural network model can potentially use sequence of frames from the past, as opposed to just one (latest) frame to accurately predict the marker locations and help overcome any loss of information from obstructive views in a single frame.

Further, post-processing on the model predictions, such as exploiting the fixed relative order of a subset of markers to each other, may enhance the localization and help avoid false positives and duplications. Several types of neural network models can be utilized, such as a convolutional neural network with a regression head to predict the target location. For instance, a CenterNet-like model or a model from the family of Transformer architectures could be used.

Another neural network model can be trained to predict accurate pixel-level masks of the various components of the deployment system, such as the flow diverter device (e.g., stent) and the guide/delivery wires. A “mask” outlines the shape of the object identified in the image. This model can also take in a sequence of previous frames and predict the masks for the most recent frame. Given the importance of accurate predictions along the boundaries of a deployed stent, the model may be trained with specific losses, for instance, a weighted loss, to better learn the boundary of the stent. Such a weighted loss would impose a larger penalty on the network for incorrect predictions around the body lumen boundaries, during training, for example.

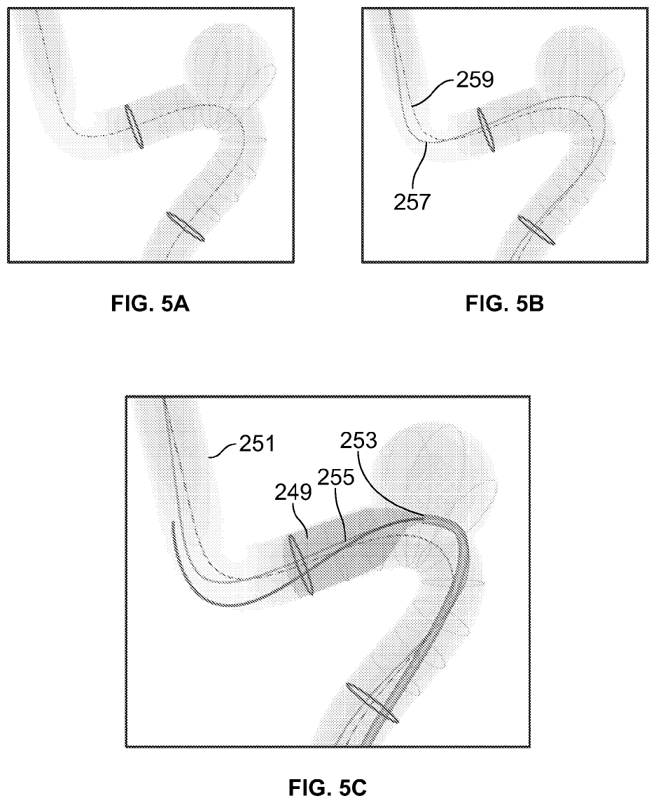

FIG. 9 illustrates generation of a mask of a wire and deployed stent using a trained model, in accordance with an example implementation. FIG. 9 depicts an image 500 representing the input frame to the model. Image 502 in the middle shows a model prediction depicting a delivery wire mask 504 and a deployed stent mask 506 identified by the trained model. Image 508 on the right represents ground truth (actual masks provided by direct observation or identified from an X-ray image) for the delivery wire and the deployed stent. As depicted, the model output shown in the image 502 is substantially accurate compared to the ground truth in the image 508.

In an example, the device deployment module 104 can further implement a model that is trained on a dataset of low- and high-resolution images with deployed stent to learn the structural properties of a stent, conditioned on the type of the stent. Such model can then be used to increase the resolution and/or the dynamic range of an X-ray image to predict a more accurate and visually enhanced version of a stent captured using a low-dosage X-ray setting.

When trained to be highly accurate, the model can additionally aid the stent segmentation model to output precise masks, especially at object/stent boundaries, to help derive secondary deployment properties of the stent such as the distance of the deployed stent to the wall of the body lumen (i.e., determine apposition), and the angle of the opening of the stent, among other properties. Image segmentation refers to annotating or assigning each pixel in an image to a single class or object, for example. The output is a mask that outlines the shape of the object in the image.

The model may be enhanced by conditionalizing it on known (physical) properties of the stent, which would help the model better represent the high-resolution output. The properties of the stent may be obtained from design files (including material properties of the wires of the stent, number of wires, and angle of wires), 3D computer aided design (CAD) model or 3D (micro) CT scans of the stent.

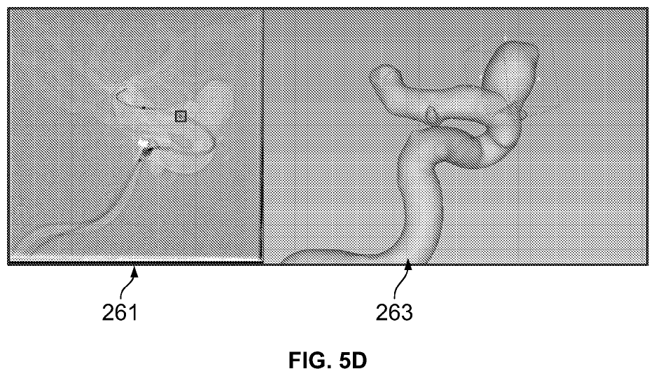

FIG. 10 illustrates a block diagram 600 representing a generative modeling workflow, in accordance with an example implementation. The block diagram may be implemented by or within the device deployment module 104.

The workflow is divided into a training phase and a testing phase. In the training phase, at block 602, the model is fed the low-dosage X-ray (low resolution) images. At block 604, the model is also fed with device (e.g., stent) and catheter generative models (e.g., CAD modes provided by manufacturers or 3D micro-CT scans). The model is then fed at block 606 with high-dose X-ray (high resolution images).

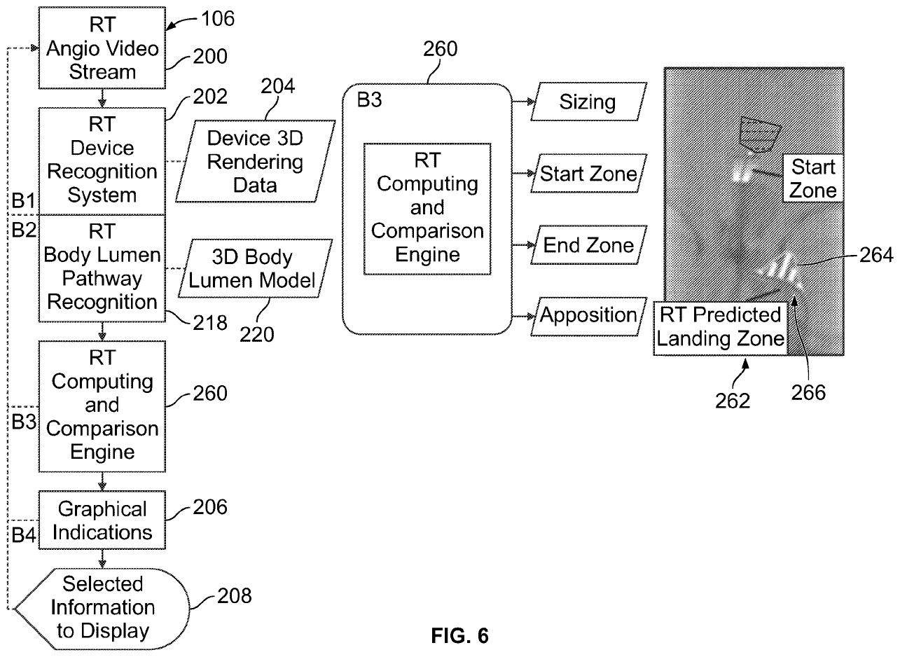

FIG. 11 illustrates a low-dose X-ray image 700 on the left and a corresponding high-dose X-ray image 702 on the right, in accordance with an example. The model can thus be trained to identify/learn the stent and its properties in the low-dosage images from the catheter and stent models and the high resolution images.

Low dose X-ray versus high dose X-ray can be a relative energy level of X-ray tube settings. For example, a low dose may be 50 milliamperage-seconds (mAs) versus a high dose of 150 mAs of the X-ray tube produced radiation settings at 120 KVp. Another example of low versus high could be 40 mAs vs 180 mAs, with 100 mAs as standard dosage. Thus, the low versus high dosage could be two relative settings in an imaging machine. In another example, low dose X-ray and high dose X-ray may involve a high imaging frame rate and a low imaging frame rate, respectively, when taking X-ray images.

Referring back to FIG. 10, during the test or inference phase, the model is fed with the low-dose image at block 608, and identifies the device (e.g., stent) and catheter at block 610. At block 612, the high-dose equivalent images are fetched to score the results of the model and make any necessary adjustments.

This way, the model is trained to identify the body lumen and device parameters using low-dose X-ray images (low resolution images), thereby eliminating or diminishing the need for high-dose X-ray imaging which can be harmful to the patient due to the radiation exposure. Further, using low resolution images can enhance computational efficiency and real-time processing of the video stream.

From the extracted segmentation masks and marker locations identified by the model, the device deployment module 104 is configured to propose various relevant indicators such as closeness of the (deployed) device to the body lumen walls (apposition), candidate locations for starting the device deployment, and other risk scores relevant for clinical decision support.

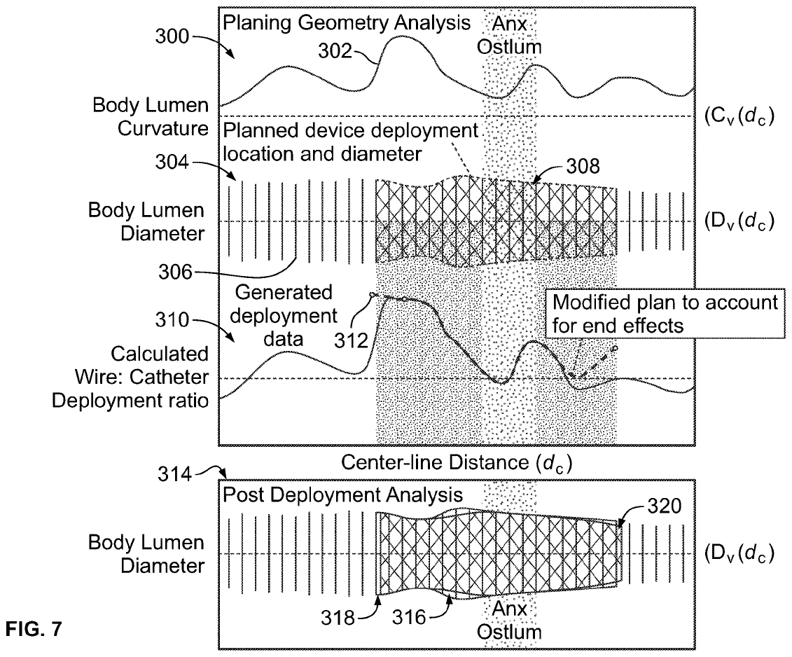

FIG. 12 is a block diagram of a computing device 800, in accordance with an example implementation. The computing device 800 can represent, or can be included in, any of the devices described above (e.g., the image-capture device 102, the device deployment module 104, the display device 106, etc.).

The computing device 800 may have processor(s) 802, a communication interface 804, and data storage 806, each connected to a communication bus 812. The computing device 800 may also include hardware to enable communication within the computing device 800 and between the computing device 800 and other devices. The hardware may include transmitters, receivers, and antennas, for example

The communication interface 804 may be a wireless interface and/or one or more wireline interfaces that allow for both short-range communication and long-range communication to one or more networks or to one or more remote devices (e.g., to allow communication with the communication bus 812). Such wireless interfaces may provide for communication under one or more wireless communication protocols, Bluetooth, Wi-Fi (e.g., an institute of electrical and electronic engineers (IEEE) 802.11 protocol), Long-Term Evolution (LTE), cellular communications, near-field communication (NFC), and/or other wireless communication protocols. Wireline interfaces may include an Ethernet interface, a CAN network interface, a USB interface, or similar interface to communicate via a wire, a twisted pair of wires, a coaxial cable, an optical link, a fiber-optic link, or other physical connection to a wireline network.

The data storage 806 may include or take the form of one or more computer-readable storage media that can be read or accessed by the processor(s) 802. The computer-readable storage media can include volatile and/or non-volatile storage components, such as optical, magnetic, organic or other memory or disc storage, which can be integrated in whole or in part with the processor(s) 802. The data storage 806 is considered non-transitory computer-readable media. In some examples, the data storage 806 can be implemented using a single physical device (e.g., one optical, magnetic, organic or other memory or disc storage unit), while in other examples, the data storage 806 can be implemented using two or more physical devices.

The data storage 806 is thus a non-transitory computer readable storage medium, and executable instructions 814 are stored thereon. The executable instructions 814 include computer executable code. When the executable instructions 814 are executed by the processor(s) 802, the processor(s) 802 are caused to perform operations of the computing device 800 (e.g., operations performed by the image-capture device 102, the device deployment module 104, or the display device 106).

The processor(s) 802 may be a general-purpose processor or a special purpose processor (e.g., digital signal processors, application-specific integrated circuits (ASIC), etc.). The processor(s) 802 may receive inputs from the communication interface 804, and process the inputs to generate outputs that are stored in the data storage 806. The processor(s) 802 can be configured to execute the executable instructions 814 (e.g., computer-readable program instructions) that are stored in the data storage 806 and are executable to provide the functionality of the computing device 800 described herein.

The computing device 800 can further include an output interface 808 to output information to other devices. If the computing device 800 represents the display device 106, it further includes a display 810. The output interface 808 outputs information to the display 810 or to other components as well. Thus, the output interface 808 can be a wireless interface (e.g., transmitter) or a wired interface as well. The processor(s) 802 may receive inputs from the communication interface 804, and process the inputs to generate outputs to the display 810.

In another example, the output interface 808 can output information in electronic forms to provide feedback or command to a robotic interface 816 controlling the delivery of devices via various control means to allow a robot to change and operate linear or rotational position of devices.

The output interface 808 can also, at the same time, provide relevant information and feedback to in-person and remote proctors via feedback mechanisms 818 (e.g., visual or audiovisual feedback mechanisms). The feedback mechanisms 818 could include displays, extended devices, hologram devices, virtual/augmented reality wearables or headsets, visual dashboard on screen, audio sounds, audio feedback wearables, tactile sensory devices, and similar sensory feedback mechanisms.

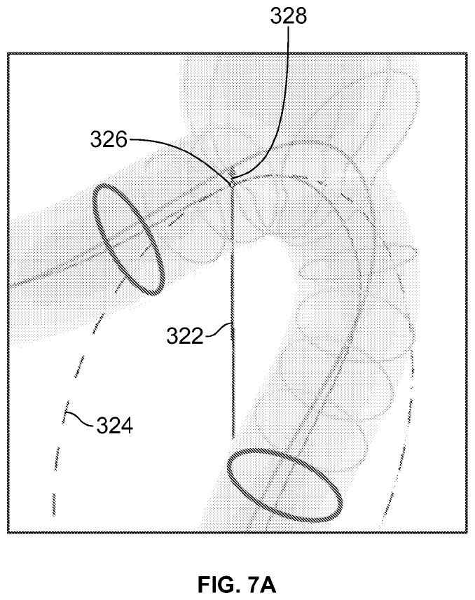

FIG. 13 is a flowchart of a method 900 for augmenting a real-time intraoperative X-ray video feed with anatomical and device related overlays and metrics, in accordance with an example implementation. The method 900 can, for example, be performed by the device deployment module 104.

The method 900 may include one or more operations, or actions as illustrated by one or more of blocks 902-910, 1000, 1100, 1200, 1300, 1400, 1500, 1600, 1700, and 1800-1806. Although the blocks are illustrated in a sequential order, these blocks may in some instances be performed in parallel, and/or in a different order than those described herein. Also, the various blocks may be combined into fewer blocks, divided into additional blocks, and/or removed based upon the desired implementation.

In addition, for the method 900 and other processes and operations disclosed herein, the flowchart shows operation of one possible implementation of present examples. In this regard, each block may represent a module, a segment, or a portion of program code, which includes one or more instructions executable by processors for implementing specific logical operations or steps in the process. The program code may be stored on any type of computer readable medium or memory, for example, such as a storage device including a disk or hard drive. The computer readable medium may include a non-transitory computer readable medium or memory, for example, such as computer-readable media that stores data for short periods of time like register memory, processor cache and Random Access Memory (RAM). The computer readable medium may also include non-transitory media or memory, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, a tangible storage device, or other article of manufacture, for example. In addition, for the method 900 and other processes and operations disclosed herein, one or more blocks in FIG. 12 may represent circuitry or digital logic that is arranged to perform the specific logical operations in the process.

At block 902, the method 900 includes receiving, at a processor (e.g., processor(s) 802 of the device deployment module 104) in real-time, a video stream of an interventional procedure captured by the image-capture device 102 of a body lumen and a device being deployed within the body lumen.

At block 904, the method 900 includes identifying, by the processor in the video stream, the body lumen and the device being deployed within the body lumen.

At block 906, the method 900 includes visually superimposing, by the processor on the video stream in real-time, body lumen markers indicating characteristics of the body lumen, as shown in FIGS. 3, 5, for example. In an example, the user may have the ability to turn on and off the superimposition of the body lumen markers or any other markers on the display via a user interface (e.g., using graphical user interface items such as menus, buttons, etc.) as the user desires

At block 908, the method 900 includes visually presenting, by the processor on the video stream in real-time, a display of device markers indicating position of the device in real-time during deployment as shown in FIGS. 2, 4, 6, 8, for example.

At block 910, the method 900 includes providing, by the processor on the video stream in real-time, visual indicators of parameters of the device including apposition of the device as shown in FIGS. 4, 7, for example.

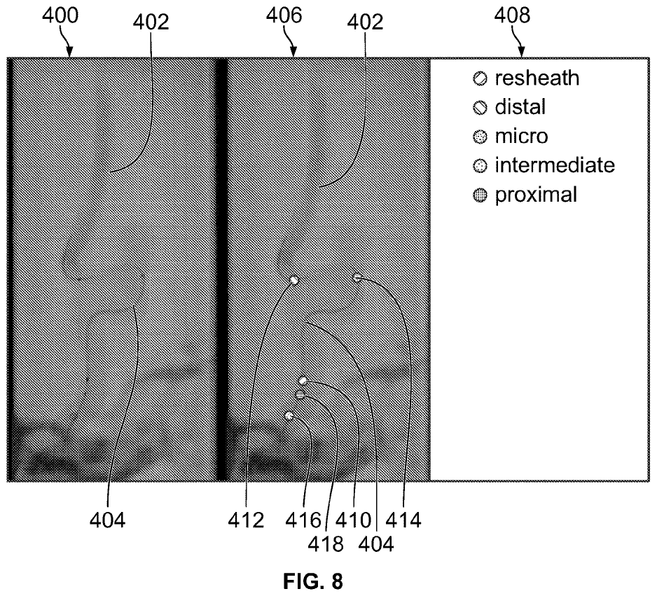

FIG. 14 is a flowchart of additional operations that are executable with the method 900, in accordance with an example implementation. The body lumen markers may include a curvature marking. At block 1000, the operations include visually marking one or more sections of the body lumen where curvature of the body lumen exceeds a threshold curvature to indicate tortuosity of the body lumen to a surgeon performing the intervention procedure as shown by the curvature markings 228-230 in FIG. 3, and the curvature markings 254, 256 in FIG. 5, for example.

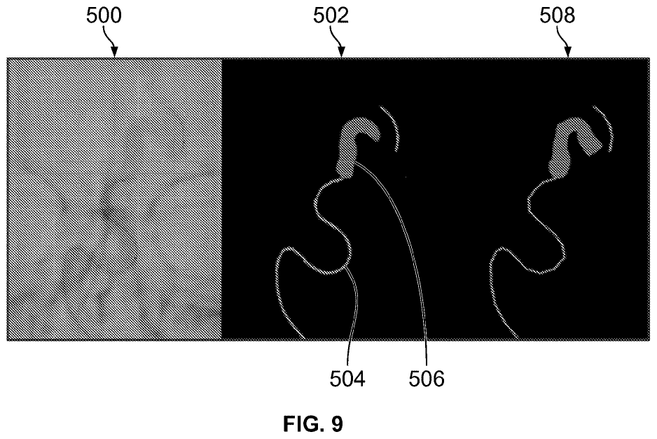

FIG. 15 is a flowchart of additional operations that are executable with the method 900, in accordance with an example implementation. The body lumen markers may include a no-start and/or a no-end zone that should be avoided when deploying the device. At block 1100, the operations include visually marking one or more sections of the body lumen with bifurcation or hidden branches to inform a surgeon during deployment of the device that the one or more sections should be avoided as a location where ends of the device should be deployed, as shown in FIG. 3 by the no-start zone 232, in FIG. 5 by the no-start zone 258, and in FIG. 6 by the no-start zone 268.

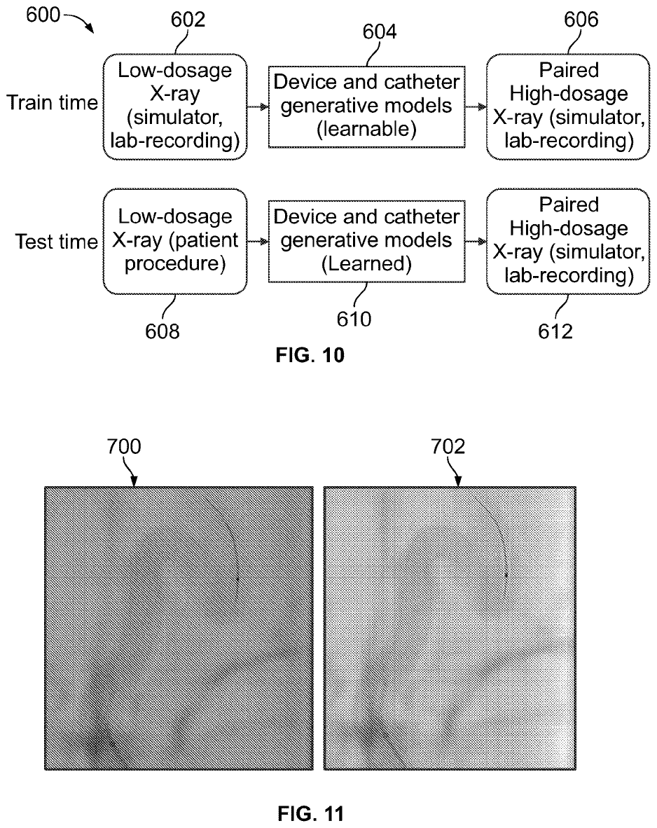

FIG. 16 is a flowchart of additional operations that are executable with the method 900, in accordance with an example implementation. At block 1200, the operations include visually marking, by the processor on the video stream in real-time, markers indicating a catheter and wire used to deploy the device, as shown in FIG. 2, 8, for example.

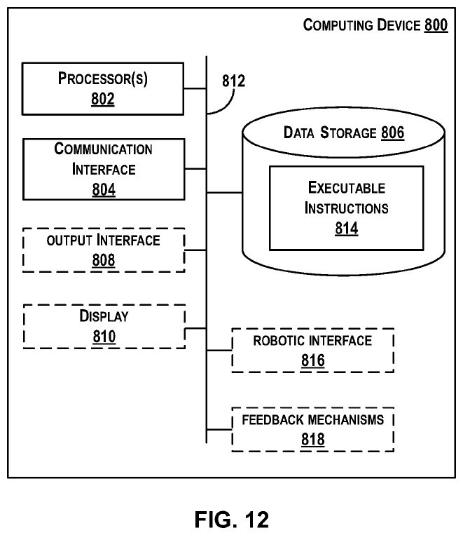

FIG. 17 is a flowchart of additional operations that are executable with the method 900, in accordance with an example implementation. At block 1300, the operations include visually presenting indicators of a proximal end and a distal end of the device, as shown in FIG. 6, for example.

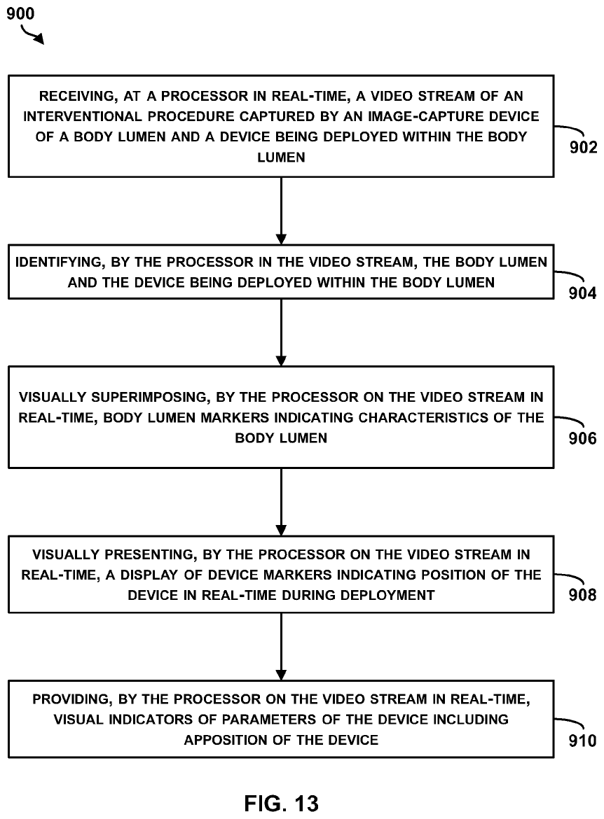

FIG. 18 is a flowchart of additional operations that are executable with the method 900, in accordance with an example implementation. At block 1400, the operations include visually displaying, by the processor on the video stream in real-time, a graphical representation of a landing probability indicating a probability that the proximal end of the device would be disposed at a particular location when deployment is completed, as shown in FIG. 6, for example. In an example, the processor determines the probability taking into consideration foreshortening effects to determine a final length of the device as described above.

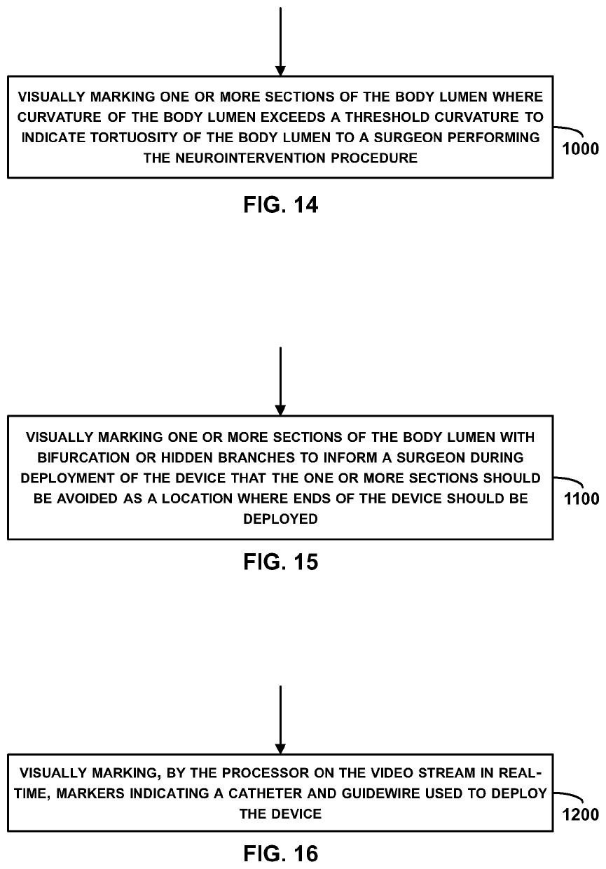

FIG. 19 is a flowchart of additional operations that are executable with the method 900, in accordance with an example implementation. The processor has access to a three-dimensional (3D) model of the device in 3D space (as described above with respect to the block 204, for example). At block 1500, the operations include visually presenting the display of device markers indicating the position of the device in real-time during deployment based on the 3D model of the device as shown in FIGS. 2, 4, 6, for example.

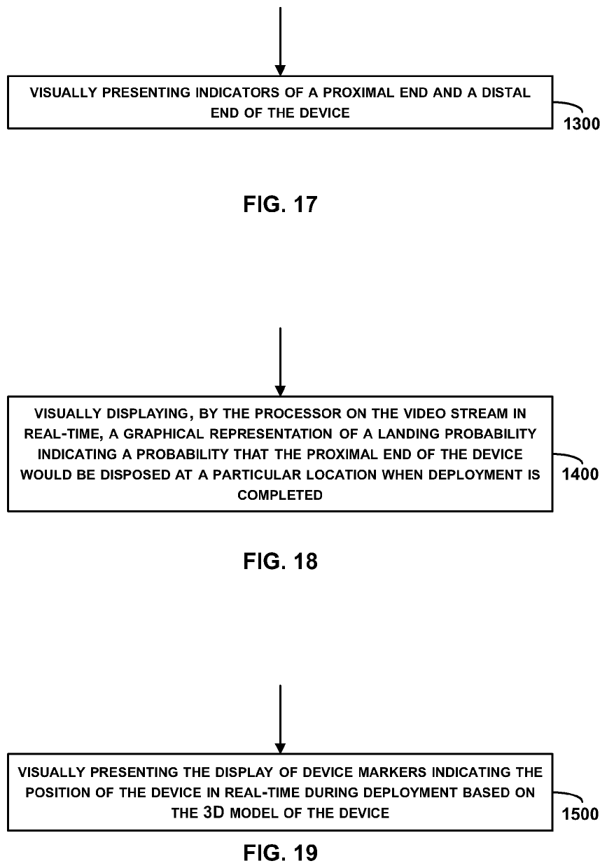

FIG. 20 is a flowchart of additional operations that are executable with the method 900, in accordance with an example implementation. The processor has access to a 3D body lumen model of the body lumen generated from images collected prior to the intervention procedure via the image-capture device (as described above with respect to the block 220, for example). At block 1600, the operations include visually superimposing the body lumen markers indicating characteristics of the body lumen based on the 3D body lumen model, as shown in FIGS. 3, 5, for example.

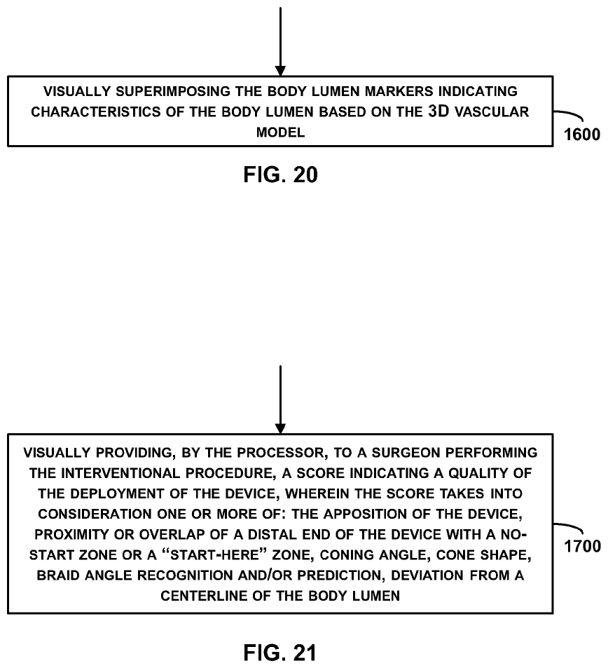

FIG. 21 is a flowchart of additional operations that are executable with the method 900, in accordance with an example implementation. At block 1700, the operations include visually providing, by the processor, to a surgeon performing the interventional procedure, a score indicating a quality of the deployment of the device, wherein the score takes into consideration one or more of: the apposition of the device, proximity or overlap of a distal end of the device with a no-start zone or a “start-here” zone, coning angle, cone shape, braid angle recognition and/or prediction, deviation from a centerline of the body lumen.

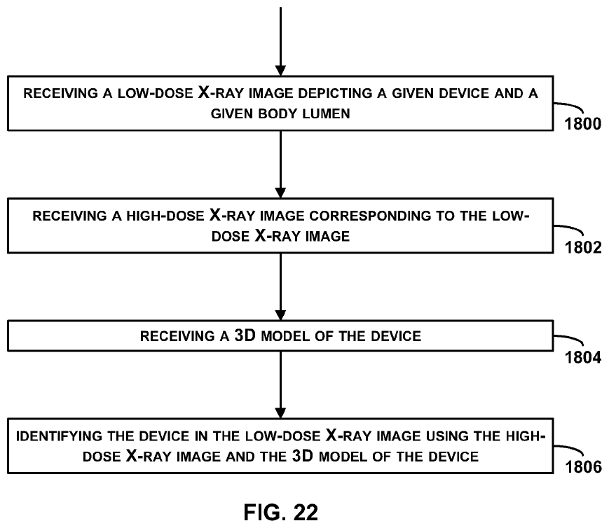

FIG. 22 is a flowchart of additional operations that are executable with the method 900, in accordance with an example implementation. In an example, as described above with respect to FIGS. 10-11, the video stream comprises a feed of low-dose X-ray images, and the processor includes a neural network model trained to identify the device and the body lumen using the low-dose X-ray images.

Particularly, at block 1800, the operations include receiving a low-dose X-ray image (the block 602 and the low-dose X-ray image 700) depicting a given device and a given body lumen. At block 1802, the operations include receiving a high-dose X-ray image (the block 606 and the high-dose X-ray image 702) corresponding to the low-dose X-ray image. At block 1804, the operations include receiving a 3D model of the device (the block 604). At block 1806, the operations include identifying the device in the low-dose X-ray image using the high-dose X-ray image and the 3D model of the device.

The detailed description above describes various features and operations of the disclosed systems with reference to the accompanying figures. The illustrative implementations described herein are not meant to be limiting. Certain aspects of the disclosed systems can be arranged and combined in a wide variety of different configurations, all of which are contemplated herein.

Further, unless context suggests otherwise, the features illustrated in each of the figures may be used in combination with one another. Thus, the figures should be generally viewed as component aspects of one or more overall implementations, with the understanding that not all illustrated features are necessary for each implementation.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

Further, devices or systems may be used or configured to perform functions presented in the figures. In some instances, components of the devices and/or systems may be configured to perform the functions such that the components are actually configured and structured (with hardware and/or software) to enable such performance. In other examples, components of the devices and/or systems may be arranged to be adapted to, capable of, or suited for performing the functions, such as when operated in a specific manner.

By the term “substantially” or “about” it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide.

The arrangements described herein are for purposes of example only. As such, those skilled in the art will appreciate that other arrangements and other elements (e.g., machines, interfaces, operations, orders, and groupings of operations, etc.) can be used instead, and some elements may be omitted altogether according to the desired results. Further, many of the elements that are described are functional entities that may be implemented as discrete or distributed components or in conjunction with other components, in any suitable combination and location.

While various aspects and implementations have been disclosed herein, other aspects and implementations will be apparent to those skilled in the art. The various aspects and implementations disclosed herein are for purposes of illustration and are not intended to be limiting, with the true scope being indicated by the following claims, along with the full scope of equivalents to which such claims are entitled. Also, the terminology used herein is for the purpose of describing particular implementations only, and is not intended to be limiting.

Embodiments of the present disclosure can thus relate to one of the enumerated example embodiments (EEEs) listed below.

EEE 1 is a method comprising: receiving, at a processor in real-time, a video stream of an interventional procedure captured by an image-capture device of a body lumen and a device being deployed within the body lumen; identifying, by the processor in the video stream, the body lumen and the device being deployed within the body lumen; visually superimposing, by the processor on the video stream in real-time, body lumen markers indicating characteristics of the body lumen; visually presenting, by the processor on the video stream in real-time, a display of device markers indicating position of the device in real-time during deployment; and providing, by the processor on the video stream in real-time, visual indicators of parameters of the device.

EEE 2 is the method of EEE 1, wherein the body lumen markers include a curvature marking, size, and location of the body lumen, and wherein visually superimposing the body lumen markers including the curvature marking comprises: visually marking one or more sections of the body lumen where curvature of the body lumen exceeds a threshold curvature to indicate tortuosity of the body lumen to a surgeon performing the interventional procedure.

EEE 3 is the method of any of EEEs 1-2, wherein the body lumen markers include a no-start zone and/or a no-end zone that should be avoided when deploying the device, and wherein visually superimposing the body lumen markers comprises: visually marking one or more sections of the body lumen with bifurcation or hidden branches to inform a surgeon during deployment of the device that the one or more sections should be avoided as a location where ends of the device should be deployed.

EEE 4 is the method of any of EEEs 1-3, further comprising: visually marking, by the processor on the video stream in real-time, markers indicating a catheter and wire used to deploy the device.

EEE 5 is the method of EEE 4, further comprising: visually presenting information indicating whether the catheter follows a preferred catheter track guideline.

EEE 6 is the method of any of EEEs 1-5, wherein visually presenting, by the processor on the video stream in real-time, the display of the device markers comprises: visually presenting indicators of a proximal end and a distal end of the device.

EEE 7 is the method of EEE 6, further comprising: visually displaying, by the processor on the video stream in real-time, a graphical representation of a landing probability indicating a probability that the proximal end of the device would be disposed at a particular location when deployment is completed.

EEE 8 is the method of EEE 7, wherein determining the probability takes into consideration foreshortening effects to determine a final length of the device.

EEE 9 is the method of any of EEEs 1-8, wherein the processor has access to a three-dimensional (3D) model of the device in 3D space, and wherein visually presenting the display of device markers indicating the position of the device in real-time during deployment is based on the 3D model of the device.

EEE 10 is the method of any of EEEs 1-9, wherein the processor has access to a 3D body lumen model of the body lumen generated from previously collected images via the image-capture device, and wherein visually superimposing the body lumen markers indicating characteristics of the body lumen is based on the 3D body lumen model.

EEE 11 is the method of any of EEEs 1-10, further comprising: generating, by the processor, a score indicating a quality of the deployment of the device, wherein the score takes into consideration one or more of: apposition of the device, proximity or overlap of a distal end of the device with a no-start zone or a “start-here” zone, coning angle, cone shape, braid angle and braid density recognition and/or prediction, deviation from a defined path including centerline of ideal deployment path, of the body lumen; and providing feedback indicative of the score to (i) a user via audiovisual feedback, virtual reality, or augmented reality display, or (ii) a robotic interface controlling delivery of the device to allow a robot to change linear or rotational position of the device.

EEE 12 is the method of any of EEEs 1-11, wherein the video stream comprises a feed of low-dose X-ray images, wherein the processor comprises a neural network model trained to identify the device and the body lumen using the low-dose X-ray images.

EEE 13 is the method of EEE 12, wherein the neural network model is trained by: receiving a low-dose X-ray image depicting a given device and a given body lumen; receiving a high-dose X-ray image corresponding to the low-dose X-ray image; receiving a 3D model of the device; and identifying the device in the low-dose X-ray image using the high-dose X-ray image and the 3D model of the device.

EEE 14 is a system comprising: an image-capture device configured to capture in real-time a video stream of a interventional procedure involving a body lumen and a device being deployed within the body lumen; a display device in communication with the image-capture device and configured to display the video stream; and a device deployment module in communication with the image-capture device and the display device, wherein the device deployment module comprises a processor and a non-transitory computer-readable medium having stored therein a plurality of executable instructions that, when executed by the processor, causes the device deployment module to perform operations comprising any of the operations of EEEs 1-13. For example, the operations can include: receiving the video stream, identifying, in the video stream, the body lumen and the device being deployed within the body lumen, visually superimposing, on the video stream displayed on the display device in real-time, body lumen markers indicating characteristics of the body lumen, visually presenting, on the video stream displayed on the display device, a display of device markers indicating position of the device in real-time during deployment, and providing, on the video stream displayed on the display device, visual indicators of parameters of the device.

EEE 15 is the system of EEE 14, wherein the body lumen markers include a curvature marking, a no-start and/or a no-end zone that should be avoided when deploying the device, and wherein visually superimposing the body lumen markers including the curvature marking comprises: visually marking one or more sections of the body lumen where curvature of the body lumen exceeds a threshold curvature to indicate tortuosity of the body lumen to a surgeon performing the interventional procedure; and visually marking respective one or more sections of the body lumen with bifurcation or hidden branches to inform the surgeon during deployment of the device that the respective one or more sections should be avoided as a location where a proximal end of the device should be deployed.

EEE 16 is the system of any of EEEs 14-15, wherein visually presenting, on the video stream in real-time, the display of the device markers comprises: visually presenting indicators of a proximal end and a distal end of the device; and visually displaying, on the video stream in real-time, a graphical representation of a landing probability indicating a probability that the proximal end of the device would be disposed at a particular location when deployment is completed, wherein determining the probability takes into consideration foreshortening effects to determine a final length of the device.

EEE 17 is the system of any of EEEs 14-16, wherein the device deployment module has access to (i) a three-dimensional (3D) model of the device in 3D space, and (ii) a 3D body lumen model of the body lumen generated from previously collected images via the image-capture device, and wherein: visually presenting the display of device markers indicating the position of the device in real-time during deployment is based on the 3D model of the device, and visually superimposing the body lumen markers indicating characteristics of the body lumen is based on the 3D body lumen model.

EEE 18 is the system of any of EEEs 14-17, wherein the operations further comprise: generating a score indicating a quality of the deployment of the device, wherein the score takes into consideration one or more of: apposition of the device, proximity or overlap of a distal end of the device with a no-start zone or a “start-here” zone, coning angle, cone shape, braid angle and braid density recognition and/or prediction, deviation from a defined path including centerline of ideal deployment path, of the body lumen; and providing feedback indicative of the score to (i) a user via audiovisual feedback, virtual reality, or augmented reality display, or (ii) a robotic interface controlling delivery of the device to allow a robot to change linear or rotational position of the device.

EEE 19 is the system of any of EEEs 14-18, wherein the video stream comprises a feed of low-dose X-ray images, wherein the device deployment module comprises a neural network model trained to identify the device and the body lumen using the low-dose X-ray images.

EEE 20 is the system of EEE 19, wherein the neural network model is trained by: receiving a low-dose X-ray image depicting a given device and a given body lumen; receiving a high-dose X-ray image corresponding to the low-dose X-ray image; receiving a 3D model of the device; and identifying the device in the low-dose X-ray image using the high-dose X-ray image and the 3D model of the device.

EEE 21 is a method comprising: receiving, at a processor in real-time, a video stream of an interventional procedure captured by an image-capture device of a body lumen and a device being deployed within the body lumen; identifying, by the processor in the video stream, the body lumen and the device being deployed within the body lumen; visually presenting, by the processor, body lumen markers indicating characteristics of the body lumen; visually presenting, by the processor, a display of device markers indicating position of the device in real-time during deployment; and providing, by the processor on the video stream in real-time, visual indicators of parameters of the device.

EEE 22 is the method of EEE 21, further comprising: generating a display of circumferential rings denoting wall of the body lumen.

The method of EEE 21 can also include any of the other operations or steps of EEEs 1-13.

EEE 23 is a method comprising: receiving, at a processor in real-time, a video stream of an interventional procedure captured by an image-capture device of a body lumen and a device being deployed within the body lumen; identifying, by the processor in the video stream, the body lumen and the device being deployed within the body lumen; visually presenting, by the processor, a display of device markers indicating position of the device in the body lumen in real-time during deployment; and providing, by the processor on the video stream in real-time, visual indicators of parameters of the device.

EEE 24 is the method of EEE 23, further comprising: visually presenting, by the processor, body lumen markers indicating characteristics of the body lumen.

EEE 25 is the method of EEE 24, wherein visually presenting the body lumen markers indicating characteristics of the body lumen comprises: visually superimposing, by the processor on the video stream in real-time, the body lumen markers indicating characteristics of the body lumen.

The method of EEE 23 can also include any of the other operations or steps of EEEs 1-13.

The system of EEE 14 can also execute any of the operations of EEEs 21-22 and EEEs 23-25.

What is claimed is:

1. A method comprising:
receiving, at a processor in real-time, a video stream of an interventional procedure captured by an image-capture device of a body lumen and a device being deployed within the body lumen;
identifying, by the processor in the video stream, the body lumen and the device being deployed within the body lumen;
visually superimposing, by the processor on the video stream in real-time, body lumen markers indicating characteristics of the body lumen;
visually presenting, by the processor on the video stream in real-time, a display of device markers indicating position of the device in real-time during deployment; and
providing, by the processor on the video stream in real-time, visual indicators of parameters of the device.

2. The method of claim 1, wherein the body lumen markers include a curvature marking, size, and location of the body lumen, and wherein visually superimposing the body lumen markers including the curvature marking comprises:
visually marking one or more sections of the body lumen where curvature of the body lumen exceeds a threshold curvature to indicate tortuosity of the body lumen to a surgeon performing the interventional procedure.

3. The method of claim 1, wherein the body lumen markers include a no-start zone and/or a no-end zone that should be avoided when deploying the device, and wherein visually superimposing the body lumen markers comprises:
visually marking one or more sections of the body lumen with bifurcation or hidden branches to inform a surgeon during deployment of the device that the one or more sections should be avoided as a location where ends of the device should be deployed.

4. The method of claim 1, further comprising:
visually marking, by the processor on the video stream in real-time, markers indicating a catheter and wire used to deploy the device.

5. The method of claim 4, further comprising:
visually presenting information indicating whether the catheter follows a preferred catheter track guideline.

6. The method of claim 1, wherein visually presenting, by the processor on the video stream in real-time, the display of the device markers comprises:
visually presenting indicators of a proximal end and a distal end of the device.

7. The method of claim 6, further comprising:
visually displaying, by the processor on the video stream in real-time, a graphical representation of a landing probability indicating a probability that the proximal end of the device would be disposed at a particular location when deployment is completed.

8. The method of claim 7, wherein determining the probability takes into consideration foreshortening effects to determine a final length of the device.

9. The method of claim 1, wherein the processor has access to a three-dimensional (3D) model of the device in 3D space, and wherein visually presenting the display of device markers indicating the position of the device in real-time during deployment is based on the 3D model of the device.

10. The method of claim 1, wherein the processor has access to a 3D body lumen model of the body lumen generated from previously collected images via the image-capture device, and wherein visually superimposing the body lumen markers indicating characteristics of the body lumen is based on the 3D body lumen model.

11. The method of claim 1, further comprising:
generating, by the processor, a score indicating a quality of the deployment of the device, wherein the score takes into consideration one or more of: apposition of the device, proximity or overlap of a distal end of the device with a no-start zone or a “start-here” zone, coning angle, cone shape, braid angle and braid density recognition and/or prediction, deviation from a defined path including centerline of ideal deployment path, of the body lumen; and
providing feedback indicative of the score to (i) a user via audiovisual feedback, virtual reality, or augmented reality display, or (ii) a robotic interface controlling delivery of the device to allow a robot to change linear or rotational position of the device.

12. The method of claim 1, wherein the video stream comprises a feed of low-dose X-ray images, wherein the processor comprises a neural network model trained to identify the device and the body lumen using the low-dose X-ray images.

13. The method of claim 12, wherein the neural network model is trained by:
receiving a low-dose X-ray image depicting a given device and a given body lumen;
receiving a high-dose X-ray image corresponding to the low-dose X-ray image;
receiving a 3D model of the device; and
identifying the device in the low-dose X-ray image using the high-dose X-ray image and the 3D model of the device.

14. A system comprising:
an image-capture device configured to capture in real-time a video stream of a interventional procedure involving a body lumen and a device being deployed within the body lumen;
a display device in communication with the image-capture device and configured to display the video stream; and
a device deployment module in communication with the image-capture device and the display device, wherein the device deployment module comprises a processor and a non-transitory computer-readable medium having stored therein a plurality of executable instructions that, when executed by the processor, causes the device deployment module to perform operations comprising:
receiving the video stream,
identifying, in the video stream, the body lumen and the device being deployed within the body lumen,
visually superimposing, on the video stream displayed on the display device in real-time, body lumen markers indicating characteristics of the body lumen,
visually presenting, on the video stream displayed on the display device, a display of device markers indicating position of the device in real-time during deployment, and
providing, on the video stream displayed on the display device, visual indicators of parameters of the device.

15. The system of claim 14, wherein the body lumen markers include a curvature marking, a no-start and/or a no-end zone that should be avoided when deploying the device, and wherein visually superimposing the body lumen markers including the curvature marking comprises:
visually marking one or more sections of the body lumen where curvature of the body lumen exceeds a threshold curvature to indicate tortuosity of the body lumen to a surgeon performing the interventional procedure; and
visually marking respective one or more sections of the body lumen with bifurcation or hidden branches to inform the surgeon during deployment of the device that the respective one or more sections should be avoided as a location where a proximal end of the device should be deployed.

16. The system of claim 14, wherein visually presenting, on the video stream in real-time, the display of the device markers comprises:
visually presenting indicators of a proximal end and a distal end of the device; and
visually displaying, on the video stream in real-time, a graphical representation of a landing probability indicating a probability that the proximal end of the device would be disposed at a particular location when deployment is completed, wherein determining the probability takes into consideration foreshortening effects to determine a final length of the device.

17. The system of claim 14, wherein the device deployment module has access to (i) a three-dimensional (3D) model of the device in 3D space, and (ii) a 3D body lumen model of the body lumen generated from previously collected images via the image-capture device, and wherein:
visually presenting the display of device markers indicating the position of the device in real-time during deployment is based on the 3D model of the device, and
visually superimposing the body lumen markers indicating characteristics of the body lumen is based on the 3D body lumen model.

18. The system of claim 14, wherein the operations further comprise:
generating a score indicating a quality of the deployment of the device, wherein the score takes into consideration one or more of: apposition of the device, proximity or overlap of a distal end of the device with a no-start zone or a “start-here” zone, coning angle, cone shape, braid angle and braid density recognition and/or prediction, deviation from a defined path including centerline of ideal deployment path, of the body lumen; and
providing feedback indicative of the score to (i) a user via audiovisual feedback, virtual reality, or augmented reality display, or (ii) a robotic interface controlling delivery of the device to allow a robot to change linear or rotational position of the device.

19. The system of claim 14, wherein the video stream comprises a feed of low-dose X-ray images, wherein the device deployment module comprises a neural network model trained to identify the device and the body lumen using the low-dose X-ray images.

20. The system of claim 19, wherein the neural network model is trained by:
receiving a low-dose X-ray image depicting a given device and a given body lumen;
receiving a high-dose X-ray image corresponding to the low-dose X-ray image;
receiving a 3D model of the device; and
identifying the device in the low-dose X-ray image using the high-dose X-ray image and the 3D model of the device.

21. A method comprising:
receiving, at a processor in real-time, a video stream of an interventional procedure captured by an image-capture device of a body lumen and a device being deployed within the body lumen;
identifying, by the processor in the video stream, the body lumen and the device being deployed within the body lumen;
visually presenting, by the processor, body lumen markers indicating characteristics of the body lumen;
visually presenting, by the processor, a display of device markers indicating position of the device in real-time during deployment; and
providing, by the processor on the video stream in real-time, visual indicators of parameters of the device.

22. The method of claim 21, further comprising:
generating a display of circumferential rings denoting wall of the body lumen.

23. A method comprising:
receiving, at a processor in real-time, a video stream of an interventional procedure captured by an image-capture device of a body lumen and a device being deployed within the body lumen;
identifying, by the processor in the video stream, the body lumen and the device being deployed within the body lumen;
visually presenting, by the processor, a display of device markers indicating position of the device in the body lumen in real-time during deployment; and
providing, by the processor on the video stream in real-time, visual indicators of parameters of the device.

24. The method of claim 23, further comprising:
visually presenting, by the processor, body lumen markers indicating characteristics of the body lumen.

25. The method of claim 24, wherein visually presenting the body lumen markers indicating characteristics of the body lumen comprises:
visually superimposing, by the processor on the video stream in real-time, the body lumen markers indicating characteristics of the body lumen.
